(19) United States
(12) Patent Application Publication
(10) Pub. No.: US 2014/0142199 A1
(43) Pub. Date: May 22, 2014

(54) ABSORBABLE BRANCHED POLYESTERS AND POLYURETHANES

(71) Applicant: Bezwada Biomedical, LLC, Hillsborough, N (US)

(72) Inventor: Rao S. Bezwada, Whitehouse Station, NJ (US)

(21) Appl. No.: 14/161,026

(22) Filed: Jan. 22, 2014

Related U.S. Application Data
(62) Division of application No. 13/017,826, filed on Jan. 31, 2011.
(60) Provisional application No. 61/305,231, filed on Feb. 17, 2010.

(51) Int. Cl. C07C 265/12; C08G 18/77

(52) U.S. Cl. CPC C07C 265/12; C08G 18/771
USPC 514/772.3; 560/193; 560/180; 528/78; 523/105; 521/162; 521/52

(57) ABSTRACT

The present invention relates to the discovery of a new class of hydrolysable isocyanates, hydrolysable branched polyols and branched absorbable polyesters and polyurethanes prepared therefrom. The resultant absorbable polymers are useful for drug delivery, stents, highly porous foam, reticulated foam, tissue engineering, tissue adhesives, adhesion prevention, bone wax formulations, medical device coatings, surface modifying agents and other implantable medical devices. In addition, these absorbable polymers can have a controlled hydrolytic degradation profile.

This application is a divisional of co-pending U.S. patent application Ser. No. 13/017,826 filed on Jan. 31, 2011 which claims priority to U.S. Application No. 61/305,231, filed Feb. 17, 2010, both of which are incorporated herein in their entireties.

The present invention relates to the discovery of a new class of hydrolysable aliphatic isocyanates, hydrolysable branched polyols and absorbable polyesters and polyurethanes prepared therefrom. The resultant branched absorbable polymers are useful for drug delivery, tissue engineering, tissue adhesives, adhesion prevention, foams, highly porous foams, bone wax formulations, medical device coatings, surface modifying agents and other implantable medical devices. In addition, these absorbable polymers should have a controlled degradation profile.

### BACKGROUND OF THE INVENTION

Absorbable polymers are increasingly used in a wide range of biomedical applications including tissue engineering scaffolds, stents, stent coatings, foams, and adhesion prevention barriers. This increased utilization is, in part, a function of the transient nature of these polymers when used as biomedical implants or drug carriers. Medical devices made from bioabsorbable polymers can mitigate the inevitable and usually negative physiologic responses (e.g., fibrous encapsulation), which limit long-term device success. Hence, an array of absorbable polymers have been developed and studied in various biomedical applications. While significant research and development activity has been carried out on absorbable polymers, such polymers may suffer from performance deficiencies which are typically not fully recognized until new applications are identified and in-use testing has been carried out. As more uses for these materials are envisioned, an increased demand for absorbable polymers with new and improved properties targeted to address performance deficiencies may be expected to follow.

Polymers that are designed to degrade under physiological conditions are referred to as absorbable polymers. These polymers are sometimes also referred to as biodegradable, bioerodible, bioabsorbable, absorbable or hydrolyzable polymers. Synthetic absorbable polymers are generally classified into polyesters, polyorthoesters, polyanhydrides, polyesteramides, polyoxaesters.

Of the synthetic absorbable polymers, polyesters find numerous applications in medical, surgical and controlled delivery applications and are the key components of a majority of absorbable medical devices, ranging from sutures, staples, orthopedic screws and implantable surgical devices to tissue engineering scaffolds.

Most synthetic absorbable polyesters are produced by ring opening homo-polymerization or copolymerization of five key lactone-based, safe and biocompatible monomers. These are glycolide, L-lactide and its isomers, ε-caprolactone, p-dioxanone and trimethylenecarbonate (TMC). The ring opening (co) polymerization is carried out in the presence of a catalyst. Medical device applications of synthetic absorbable polyesters require the catalysts be highly biocompatible and non-immunogenic. Although a number of catalysts can be used from a functional standpoint, specific tin catalysts have typically been used. This is because of their functional effectiveness in addition to their biocompatibility at the levels they are used. Stannous octoate, based on 2-ethylhexanoic acid, and stannous chloride dihydrate are the most commonly used catalysts for such polymerizations.

Hydroxyl-containing compounds are typically used as an initiator in the ring opening polymerizations. These compounds have either a single hydroxyl group, such as hexanol or dodecanol, or multihydroxyl groups such as ethylene glycol (diol) or glycerol (triol). Use of initiators with more than two hydroxyl groups results in the formation of branched polymers.

These ring-opening polymerizations can be carried out in bulk (i.e., without any solvent) or in solution. However, solution polymerization is little used in production. This poor utilization may be attributed to the difficulty of removing residual solvent to the acceptable level. The majority of the absorbable (co) polyesters are commercially produced via solventless-melt, ring-opening polymerizations at high temperatures in the presence of tin catalyst. Nevertheless, solution polymerization is very useful for preparing polyesters with very low polydispersity, and allows for efficient heat removal.

Segmented polyurethane elastomers have enjoyed wide use as biomaterials generally due to their excellent mechanical properties and desirable chemical versatility. However, the vast majority of research devoted to the development of biomedical polyurethanes has focused on long-term applications such as vascular grafts and pacemaker lead insulators.

Despite progress in the general development of polyurethanes and similar polymers for use in biomedical applications, relatively little research has been directed to developing absorbable polyurethanes for temporary, rather than longer-term implantation. See Fuller et al., U.S. Pat. No. 4,829,099; Beckmann et al., U.S. Patent Publication Nos. 2005/0013793, 2004/0170597, and 2007/0014755; Bruin et al., PCT Publication No. WO 95/26762; Woodhouse et al., U.S. Pat. No. 6,221,997; Cohn et al., U.S. Pat. No. 4,826,945, which generally discuss recent advances made in the field of absorbable polyurethanes.

Subsequent work by Bruin et al., PCT No. WO 95/26762, discloses the synthesis of crosslinked polyurethane networks incorporating lactide or glycolide and ε-caprolactone joined by a lysine-based diisocyanate. Bruin discloses that these polymers display good elastomeric properties and degrade within about 26 weeks in vitro and about 12 weeks in vivo (subcutaneous implantation in guinea pigs). Despite their disclosed desirable flexibility and degradation characteristics, these highly crosslinked polymers are not extensively used in some biomedical applications because in some cases they cannot be readily processed into surgical articles, for example, using standard techniques such as solution casting or melt processing, as is the case for the more typical linear, segmented polyurethanes.

Cohn et al., EP 295055 discloses a series of elastomeric polyester-polyether-polyurethane block copolymers intended for use as surgical articles. However, these polymers may be relatively stiff and may have low tensile strength, which may preclude their use as elastomeric biomaterials. Beckmann et al., U.S. Patent Publication No. 2005/0013793 describes polyurethane-based biodegradable adhesives from multi-isocyanate functional molecules and multifunctional precursor molecules with terminal groups selected from hydroxyl and amino groups. Woodhouse et al. discloses absorbable polyurethanes derived from amino acids. However, all these absorbable polyurethanes may suffer from one or more of the following drawbacks: (a) the very slow rate of formation of polyurethane which may be attributed to the low reactivity of the polyisocyanates, and (b) the lack of tunable physical and/or mechanical properties and/or controllable hydrolytic degradation profiles for biodegradable polyisocyanates or absorbable polyurethanes derived therefrom.

Bezwada (U.S. Patent Application Publication Nos. 20060188547, 20090292029, European Patent Publication No. EP 1937182 and WO 2007030464) discloses polyurethanes, the corresponding polyisocyanates, and preparations of their manufacture and use wherein the polyurethanes and/or polyisocyanates were reported to be absorbable.

Shaped articles made from polyurethane polymers have been accepted for a variety of applications, including some biomedical applications. Generally speaking, the term “polyurethane” refers to a family of high strength, resilient synthetic polymeric materials containing recurring urethane, urea, and/or ester groups in the polymer backbone. Polyurethanes are generally prepared from isocyanates, polyols including polyether polyols and polyester polyols, and chain extenders such as 1,4-butanediol and/or 1, 2 ethylene diamine. While polyurethane polymers have certain useful properties, shaped articles based on these polymers are not typically absorbable and may therefore be unacceptable in circumstances that require bioabsorption. For example, certain biomedical applications, such as surgical devices including but not limited to monofilament and multifilament sutures, films, sheets, plates, clips, staples, pins, screws, stents, stent coatings, and the like, generally require the use of a material that is absorbable.

In addition, high strength, highly flexible, tough, and durable fibers that possess a prolonged flex fatigue life are needed for use as braided, knitted, woven, or non-woven implants to augment and/or temporarily reinforce autologous tissue grafts or to serve as scaffolds for tissue regeneration.

Other well known uses for absorbable polymers that have not been fully realized or perfected with available polymers include scaffolds for tissue engineering, bioabsorbable knitted vascular grafts, drug-releasing devices, growth factor-releasing implants for bone and tissue regeneration, and fiber-reinforced composites for orthopedic applications.

Despite advancements in the art of producing polymeric materials and methods for making polymers suitable for use in drug delivery, tissue adhesives, adhesion prevention barrier, bone wax formulations, stent coatings, scaffolds, films, molded devices, and similar surgical articles, presently available polymers generally lack adequate performance properties desirable in surgical articles, for example, those related to bioabsorption, flex fatigue life, strength in use, flexibility and/or durability. Thus, there continues to be a need for new devices and polymers having tunable physical and/or biological properties, so that medical devices and surgical articles having a variety of end uses can be prepared. The present invention is directed, among other things, to absorbable drug delivery systems, tissue adhesives, adhesion prevention barrier, bone wax formulations, coatings including stent coatings, tissue engineering scaffolds, films, molded devices and/or flexible films with tunable physical and biological properties, and improving the processability of polyurethanes during molding and extrusion, surface properties of finished products.

### SUMMARY OF THE INVENTION

Accordingly, the present invention is directed, in part, to provide novel absorbable materials that are useful for drug delivery, tissue engineering, tissue adhesives, adhesion prevention, bone wax formulations and other implantable medical devices.

In certain embodiments, the invention provides novel branched polyesters which are biodegradable and biocompatible. In certain embodiments, the invention provides novel branched polyurethanes which are biodegradable and biocompatible.

In certain embodiments, the invention provides novel branched polyurethanes and polyesters which are biodegradable and biocompatible for bone wax formulations.

In certain embodiments, the invention provides novel branched polyurethanes and polyesters which are biodegradable and biocompatible for tissue engineering, drug delivery, tissue adhesives, adhesion prevention, other implantable medical devices, foam, highly porous foam, reticulated foams, drug device combinations, medical device coatings, films, non-implantable medical device, and the like.

In certain embodiments, the invention provides novel hydrolysable isocyanates for use in the formation of branched polyurethanes.

In certain embodiments, the invention provides novel hydrolysable branched aliphatic polyols with pendant long chain alkyl groups linked to the polyol backbone via a hydrolytically degradable ester or amide linkage, for use in the formation of branched polyurethanes and/or branched polyesters.

Briefly stated, the present invention relates to the discovery of a new class of hydrolysable isocyanates, hydrolysable branched polyols and absorbable polyurethanes and polyesters prepared therefrom. The resultant absorbable polymers are useful for drug delivery, tissue engineering, tissue adhesives, adhesion prevention, bone wax formulations, medical device coatings, surface modifying agents and other implantable medical devices. In addition, these absorbable polymers should have a controlled degradation profile.

Accordingly one aspect of the present invention is to prepare branched absorbable polyurethanes from at least one compound selected from:

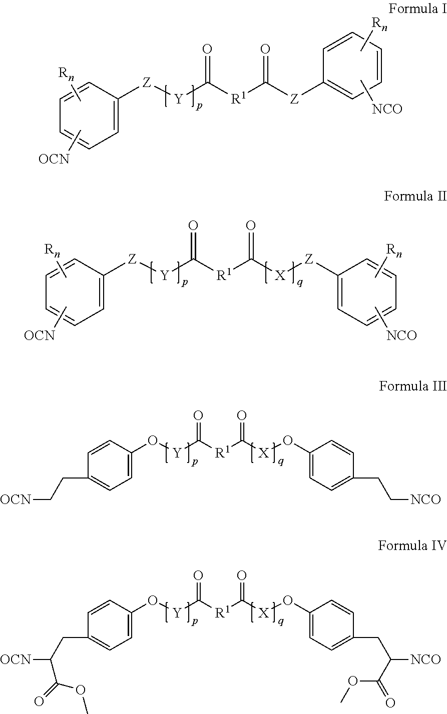

### Wherein:

each Y is independently:
   —COCH₂O— (glycolic ester moiety);
   —COCH(CH₃)O— (lactic ester moiety);
   —COCH₂OCH₂CH₂O— (dioxanone ester moiety);
   —COCH₂CH₂CH₂CH₂CH₂O— (caprolactone ester moiety);
   —CO(CH₂)_{y}O— where y is one of the numbers 2,3, 4, and 6-24 inclusive; or
   —COCH₂O(CH₂CH₂O)ₘ— where m is integer between 2-24 inclusive; and,
each X is independently:
   —OCH₂CO— (glycolic acid moiety);
   —OCH(CH₃)CO— (lactic acid moiety);
   —OCH₂CH₂OCH₂CO— (dioxanone moiety);
   —OCH₂CH₂CH₂CH₂CH₂CO— (caprolactone moiety);
   —O(CH₂)_{y}CO— or,
   —O(CH₂CH₂O)ₘCH₂CO—;
R¹ is -(D)ᵣ-, wherein r is an integer between 1-24 inclusive, or 1-20, or 1-16, or 1-10, or 1-2, wherein if r is 1 or 2, D is —CH₂—, and if r is greater, D is —CH₂— or —O—/—S— (oxygen or sulfur), wherein all individual carbons D are linked to one or two carbons, and all occurrences of —O—/—S— are linked to two carbons;
Z is —O—, —S— or —NH—;
p and q are independently integers between 0 and 6, inclusive, or from 1 to 6, or from 2 to 6, or from 1 to 4, or from 2 to 4;
each R is independently alkoxy, benzyloxy, aldehyde, halogen, carboxylic acid, NO₂ or NO₂—R⁷—, where R⁷ is lower alkyl; and
n is 0 to 4, or 0 to 3, or 0 to 2.

In certain embodiments, R¹ includes O or S. In certain embodiments, where r≧3, one or more of p or q is or both are greater than 0, or greater than 1. In certain embodiments, one or more of p or q is or both are greater than 0, or greater than 1.

All ranges herein are inclusive of the endpoints unless otherwise specified. The embodiments of the invention include those where one or both of the endpoints of a given range are not inclusive.

Another aspect of the present invention is to prepare branched absorbable polyurethanes from at least one branched hydrolysable chain extender with pendant long chain alkyl groups or biologically active compounds linked to chain extender backbone via a hydrolytically degradable ester or amide linkage selected from:

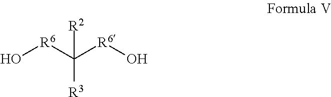

### Wherein:

R² and R³ are independently alkyl, —CO—(X)ₚ—O—R⁴ or —NH—(Y)_{q}—CO—R⁵, wherein alkyl is C6 to C20 (such as the alkyl moieties of stearic, oleic or linoleic acid), or a biologically active substance, wherein in some embodiments no more than one of R² and R³ is alkyl;
X and Y are as above;
R⁶ is -(D)_{r*}-, wherein r* is an integer between 1-24 inclusive, or 1-20, or 1-16, or 1-10, or 1-2, wherein if r* is 1 or 2, D is —CH₂—, and if r* is greater, D is —CH₂— or —O—/—S— (oxygen or sulfur), wherein all carbons of -(D)_{r*}- are linked to one or more carbons, and all occurrences of —O—/—S— are linked to two carbons;
R^{6′} is independently defined as is R⁶; p and q are as above; and
R⁴ and R⁵ are each independently is alkyl, wherein alkyl is C1 to C20 (such as the alkyl moieties of stearic, oleic or linoleic acid), a biologically active substance, or a polyethylene glycol of molecular weight from 500 to 3000 (or alkyl is C1 to C6, or C6 to C20).

In certain embodiments, one or more of p or q is or both are greater than 0, or greater than 1.

Yet another aspect of the present invention is to prepare branched absorbable polyurethanes and/or polyesters from at least one branched hydrolysable polyol with pendant long chain alkyl groups or biologically active compounds linked to polyol backbone via a hydrolytically degradable ester or amide linkage selected from

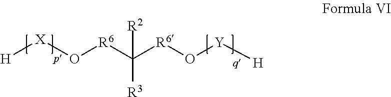

### Wherein:

R² and R³ is as above; X and Y are as above; R⁶ is as above, and R^{6′} is independently defined as is R⁶; and
p′ and q′ are independently 1-24, or 6-24, or 1-12, or 6-12 (or a range from 1 or more, 2 or more, 3 or more, 4 or more, 5 or more, or 6 or more, or from 24 or less, 18 or less 15 or less, or 12 or less).

In certain embodiments, one or more of p′ or q′ is or both are greater than 0, or greater than 1.

In certain embodiments, the invention provides surgical article or component thereof or polymeric carrier comprised of branched absorbable polyurethanes derived from isocyanates, chain extenders and polyols, wherein one or more of (A) to (C) applies: (A) the isocyanates comprise an absorbable isocyanate of Formulas I-IV; (B) the chain extenders comprise an absorbable chain extender of Formula V; or (C) the polyols comprise an absorbable polyol of Formula VI. In certain embodiments, (A) and (B) applies, or (A) and (C), or (B) and (C), or all three apply. In certain embodiments, isocyanates of Formulas I-IV comprise 5, 10, 20, 30, 40, 50, 60, 70, 80, 90 percent or more, or 100% (wt) of the isocyanate component of the polymer. In certain embodiments, chain extenders of Formula V comprise 5, 10, 20, 30, 40, 50, 60, 70, 80, 90 percent or more, or 100% (wt) of the chain extender component of the polymer. In certain embodiments, polyols of Formulas VI comprise 5, 10, 20, 30, 40, 50, 60, 70, 80, 90 percent or more, or 100% (wt) of the polyol component of the polymer.

In one embodiment branched absorbable polyesters will be prepared by reaction of branched chain extenders of Formula V and/or branched absorbable polyols of Formula VI of the present invention with diacids including but not limited to oxalic acid, succinic acid, malonic acid, butanedioic acid, adipic acid, azelaic acid, sebacic acid, diglycolic acid, 3,6-dioxaoctanedioic acid, 3,6,9-trioxaundecanoic acid, functionalized oxaacids, polyethyleneglycol diacids of average molecular weight from 300 to 2000 and blends thereof. In certain embodiments, chain extenders of Formula V comprise 5, 10, 20, 30, 40, 50, 60, 70, 80, 90 percent or more, or 100% (wt) of the chain extender component of the polymer. In certain embodiments, polyols of Formulas VI comprise 5, 10, 20, 30, 40, 50, 60, 70, 80, 90 percent or more, or 100% (wt) of the polyol component of the polymer.

In another embodiment of the present invention, branched absorbable polyesters of the present invention can be further polymerized with lactone monomers including but not limited to glycolide, lactide, caprolactone, p-dioxanone, TMC, δ-valerolactone, β-butyrolactone, morpholinedione, pivalolactone, ε-decalactone, 2,5-diketomorpholine and combinations thereof in order to control physical and biological properties.

### DETAILED DESCRIPTION OF ILLUSTRATIVE EMBODIMENT

The present invention relates to the discovery of a new class of hydrolyzable isocyanates, hydrolysable-branched polyols and absorbable polyesters and polyurethanes prepared therefrom. The absorbable polymers that result from polymerization of these absorbable isocyanates and branched chain extenders and polyols are useful for, inter alia, drug delivery, tissue engineering, tissue adhesives, adhesion prevention, bone wax formulations, medical device coatings and other implantable medical devices. In addition, these absorbable polymers should have a controlled degradation profile.

As employed above and throughout the disclosure, the terms defined below, unless otherwise indicated, shall be understood to have the defined meanings.

As used herein, unless otherwise defined “alkyl” refers to an optionally substituted, saturated straight, or branched hydrocarbon moiety having from about 1 to about 20 carbon atoms (and all combinations and subcombinations of ranges of carbon atoms and specific numbers of carbon atoms therein), or with from about 1 to about 8 carbon atoms, herein referred to as “lower alkyl”, or from about 1 to about 3 carbon atoms, such as methyl or ethyl. Alkyl groups include, but are not limited to, methyl, ethyl, n-propyl, isopropyl, n-butyl, isobutyl, t-butyl, n-pentyl, isopentyl, neopentyl, n-hexyl, isohexyl, 3-methylpentyl, 2,2-dimethylbutyl, and 2,3-dimethylbutyl.

As used herein, the term “absorbable” refers to those classes of materials that readily hydrolyze and/or enzymatically degrade upon exposure to bodily tissue in a relatively short period of time, thus experiencing a significant weight loss in that short time period. A relatively short period of time shall be judged from the context. For example, in some contexts, the relatively short period may be two weeks to eight weeks, while in others it may be eight weeks to fifty two weeks. Complete bioabsorption should take place within twelve months, or within nine months, or within six months. In this manner, the polymers derived from isocyanates of the invention may be fabricated into medical and surgical devices which are useful for a vast array of applications requiring complete or substantially complete absorption within the relatively short time periods set forth herein.

The biological properties of the absorbable polymers of the present invention used to form devices or parts thereof, as measured by its absorption rate and its breaking strength retention in vivo (BSR), can be varied to suit the needs of the particular application for which the fabricated medical device or component is intended. This can be conveniently accomplished by varying the ratio of components of the polymer chosen.

A “biologically active” substance in the context of the present invention is a substance that can act on a cell, virus, organ or organism, including but not limited to drugs (i.e. pharmaceuticals) to create a change in the functioning of the cell, virus, organ or organism. In certain embodiments of the invention, the biologically active substances are organic molecules having molecular weight of about 600 or less, or to polymeric species such as proteins, nucleic acids, and the like. A biologically active substance can be a substance used in therapy of an animal, preferably a human. For use in the invention, a biologically active substance bears, or has a functional homolog that bears, one or more hydroxyl, amino or carboxylic acid substituents, including functional derivatives such as esters, amides, methyl ethers, glycosides and other derivatives that are apparent to those skilled in the art. Examples of biologically active compounds that can be used in the present invention include Capsaicin, Vitamin E, Resveratrol and isoflavonoids.

Depending on the formation route selected, these cleavable sites may be regular along the length of the chain extender, thereby giving the segmented polyurethane or polyester a biodegradability which is, by some measure, predictable. Biodegradability is influenced by a number of factors, including crystallinity. The hydrophilicity of the polymer may also influence the degradability, that is, the extent to which water is accessible to the polymer matrix. The number of cleavage sites may also influence biodegradability. Generally speaking, the higher the number of sites, the greater the rate of degradation. Preferably, the cleavable site is an ester site and, more preferably, the cleavable ester site is derived from a hydroxy acid precursor. This provides segmented polyurethanes and polyesters with cleavable sites that may be arranged to be recognizable by enzymes.

The polyesters disclosed herein may be prepared by reacting polyols and/or chain extenders of the present invention with diacids in the presence of an organometallic catalyst at elevated temperatures. The organometallic catalyst is preferably a tin-based catalyst, e.g. stannous octoate and is present in the monomer mixture at a mole ratio of monomer-to-catalyst ranging from about 15,000 to about 80,000/1. The polymerization is typically carried out at a temperature ranging from about 120 to about 200 degree C., or about 160 to about 190 degree C., until the desired molecular weight and viscosity are achieved. The polyurethanes disclosed herein may be prepared by reacting isocyanates of the present invention with branched chain extender and polyols of the present invention and/or generic polyols including polyethylene glycols, polyesterdiols and polyetherdiols.

One of the beneficial properties of the ester elements of the polymers of the present invention is that the ester linkages are hydrolytically unstable, and therefore the polymer is absorbable because it readily breaks down into small segments when exposed to moist bodily tissue. In this regard, while it is envisioned that co-reactants could be incorporated into the reaction mixture of the dicarboxylic acid and the polyol/chain extender for the formation of the polyester pre-polymer, it is preferable that the reaction mixture does not contain a concentration of any co-reactant.

When any variable occurs more than one time in any constituent or in any formula, its definition in each occurrence is independent of its definition at every other occurrence. Combinations of substituents and/or variables are permissible only if such combinations result in stable compounds. Within the context of the present invention, compounds are stable if they do not degrade significantly prior to their intended use under normal conditions. In some instances, compounds of the invention may be designed or required to be bioabsorbed or biodegraded as part of their intended function. Absorbability and/or biodegradability, which may be an advantageous property of the present polymers, is not intended to mean that the polymeric compound are unstable.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way, including restricting it to any specific tautomeric form, except where such limit is clearly defined.

In other embodiments, the isocyanates of Formula I (for example employed in the branched absorbable polyurethanes of the present invention for applications such as described herein including without limitation stents, stent coatings, films, adhesion prevention barriers, scaffolds, foams, highly porous foams, reticulated foams, drug device combinations, medical device coatings, films, non-implantable medical devices, pharmaceutical compositions, polyurethane foams, and the like) include but are not limited to the exemplary structures (#s 1-7, respectively) shown below:

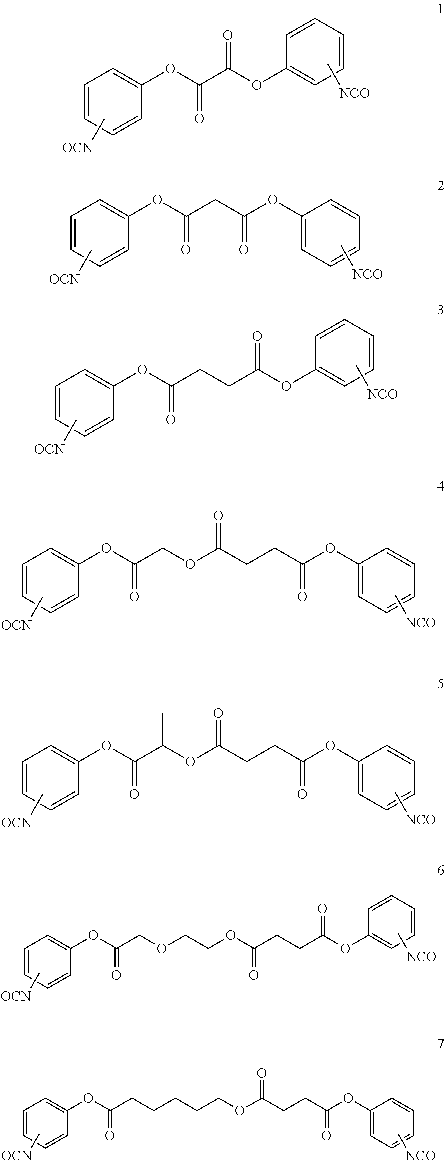

In other embodiment, the invention is directed to branched absorbable polymers (for example for such applications as described herein including without limitation stents, stent coatings, films, scaffolds, adhesion prevention barriers, highly porous foams, reticulated foams, drug device combinations, medical device coatings, films, non-implantable medical devices, polyurethane foams and pharmaceutical compositions comprising a polymeric carrier and a drug uniformly dispersed therein) wherein the polymers are derived from a hydrolysable isocyanate of Formula II, having the following structures (#s 8-11, respectively):

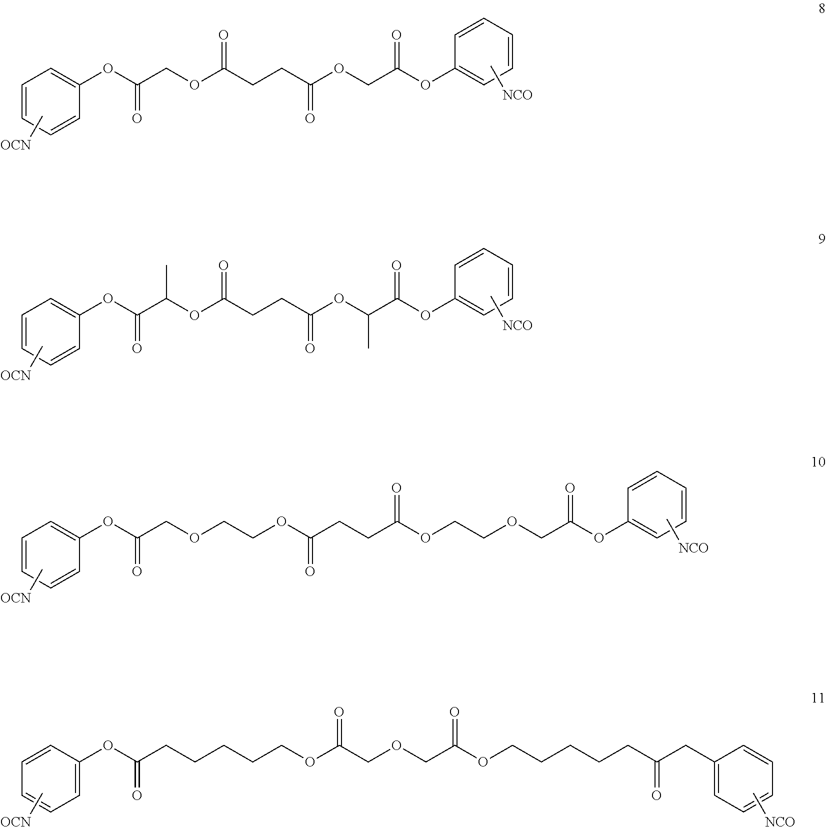

In certain embodiments directed to branched absorbable polymers (for example for such applications as described herein including without limitation stents, stent coatings, films, scaffolds, adhesion prevention barrier, polyurethane foams and pharmaceutical compositions comprising a polymeric carrier and a drug uniformly dispersed therein) wherein the polymers are derived from a hydrolysable isocyanate of Formula III having the following structures (#s 12-14, respectively):

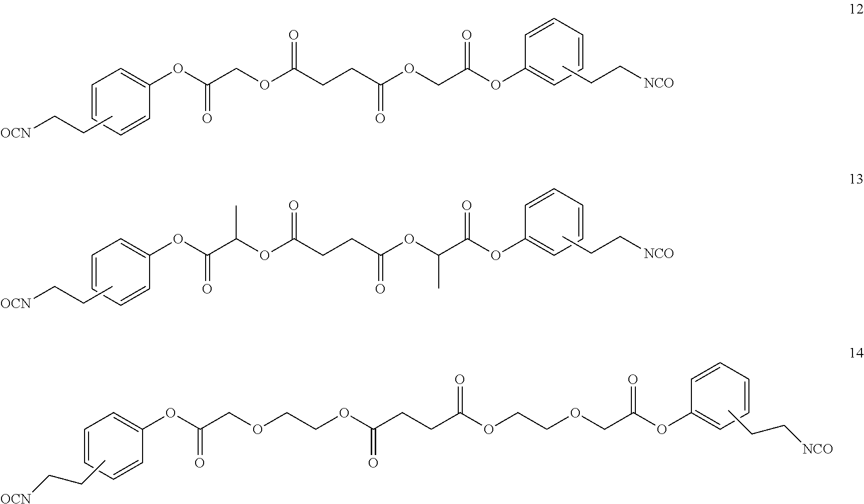

In certain embodiments, examples of isocyanates of Formula IV that may be used to prepare branched absorbable polyurethanes of the present invention polymers (for example for such applications as described herein including without limitation stents, stent coatings, films, scaffolds, adhesion prevention barrier and polyurethane foams) include but are not limited to the exemplary compounds shown below (#s 15-17, respectively):

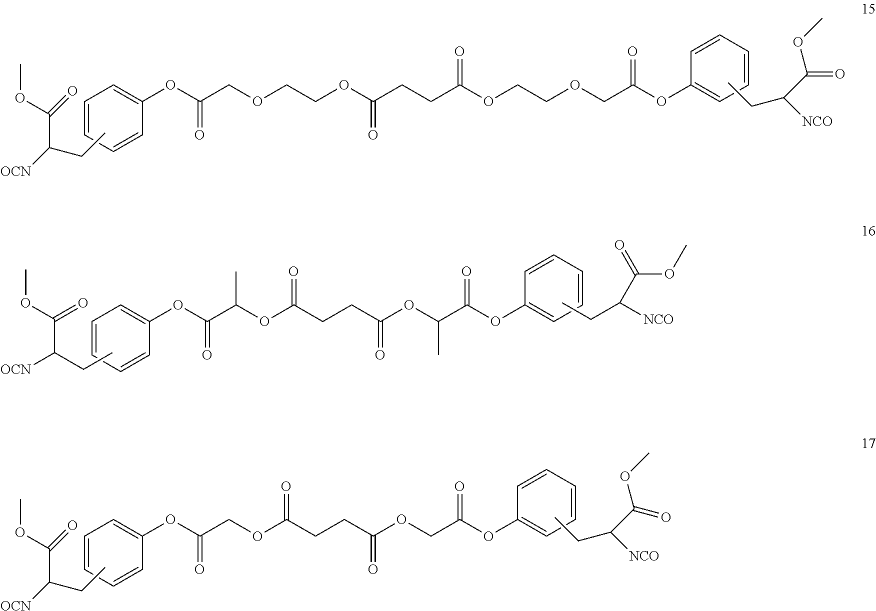

In some embodiments, the branched polyurethanes and/or their precursors of Formula I, II, III or IV have tunable physical and/or biological properties.

Suitable examples of amines that may be used to prepare hydrolysable isocyanates of the present invention in addition to branched polyureas, polyureaurethanes, polyamides, polyesteramides and epoxy amine resins include but are not limited to the exemplary compounds #s 1′-17′, which correspond to the compounds of the corresponding non-primed numbers, except the isocyanate group is replaced by amino. For example, 1 and 1′ are as follows:

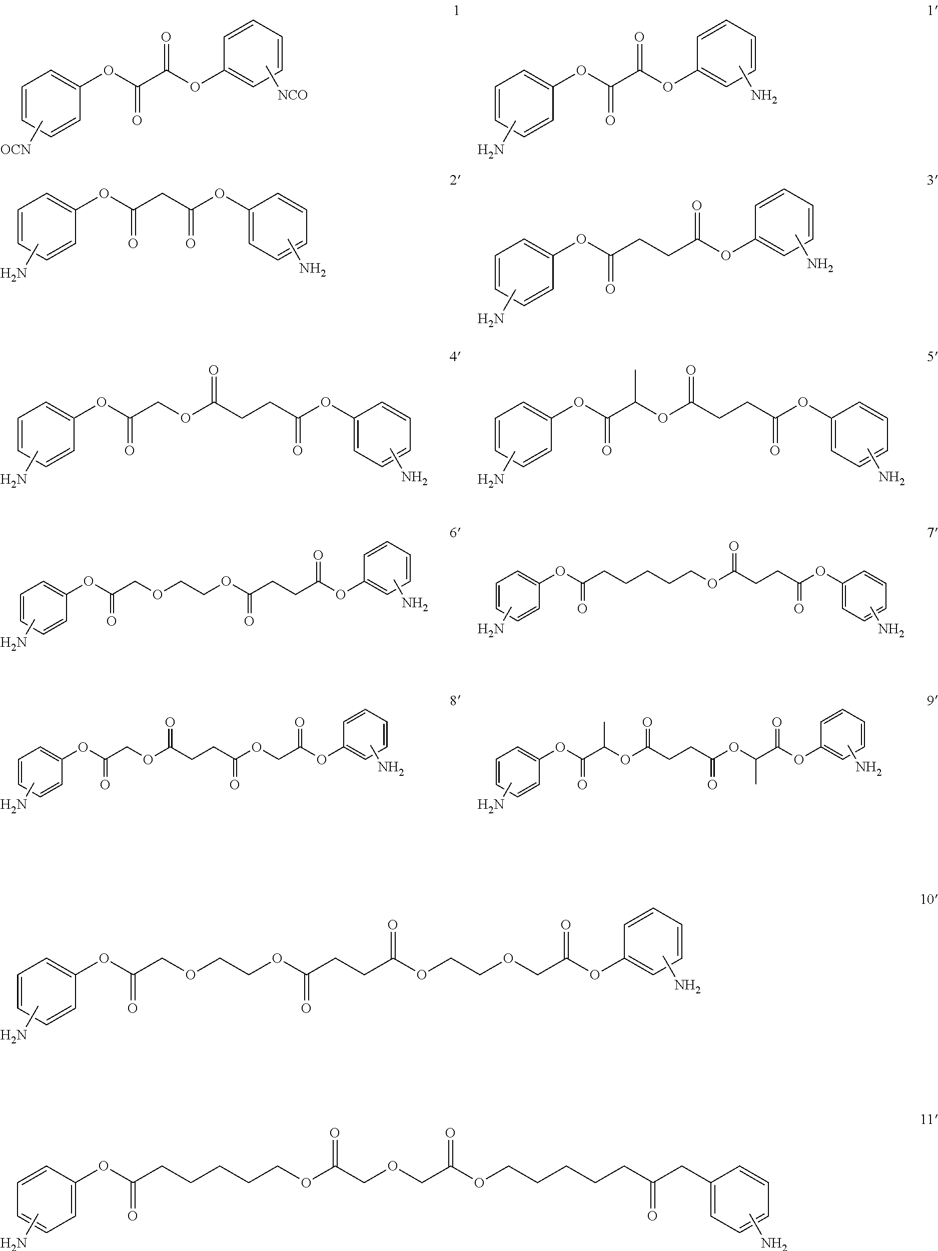

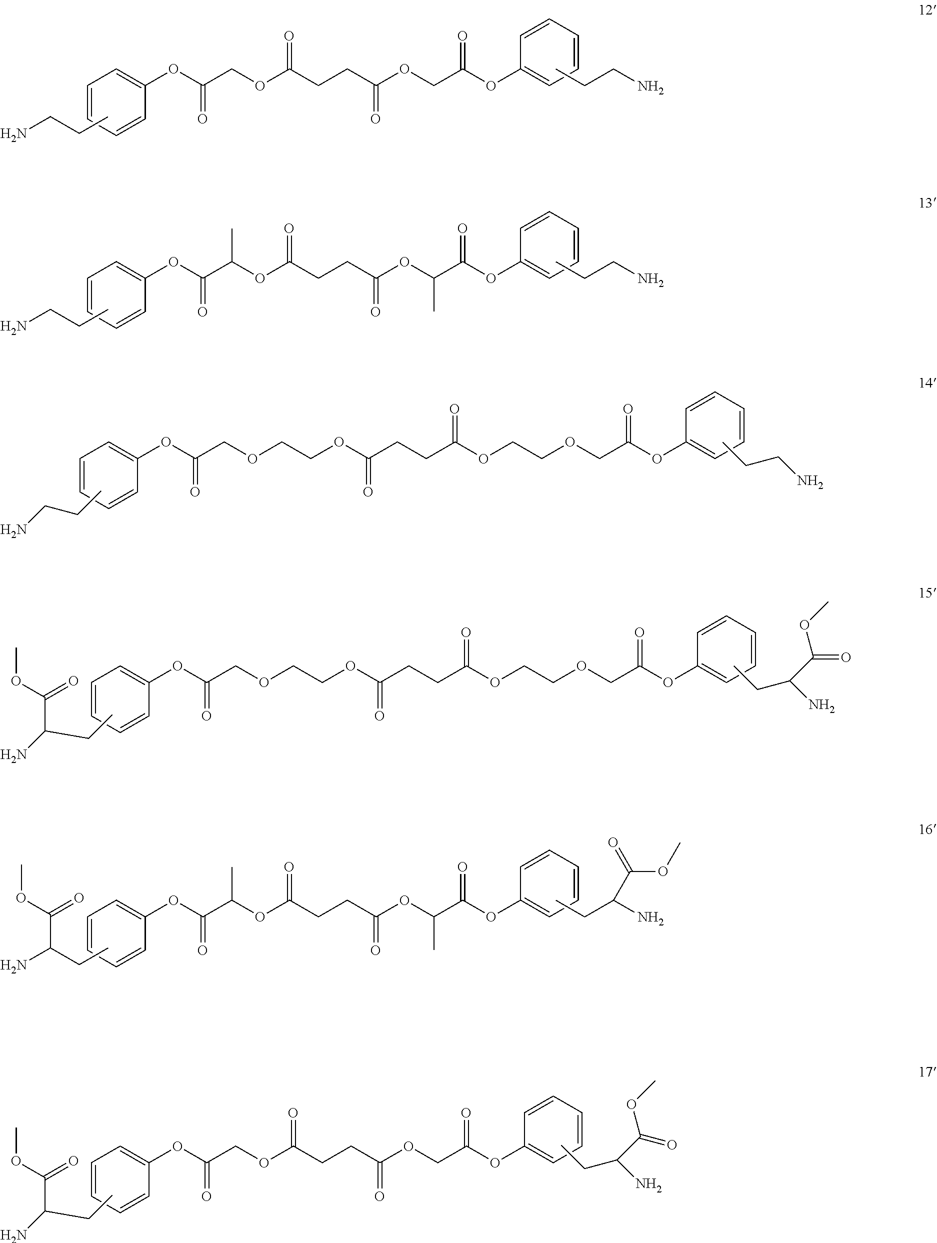

In other embodiments, branched absorbable polyurethanes of the present invention, such as in connection with their use as stents, stent coatings, films, scaffolds, adhesion prevention barrier and polyurethane foams, may be derived from isocyanates of present invention and reacting them with chain extenders of Formula V including but not limited to the examples shown below (#s V1-V13, respectively):

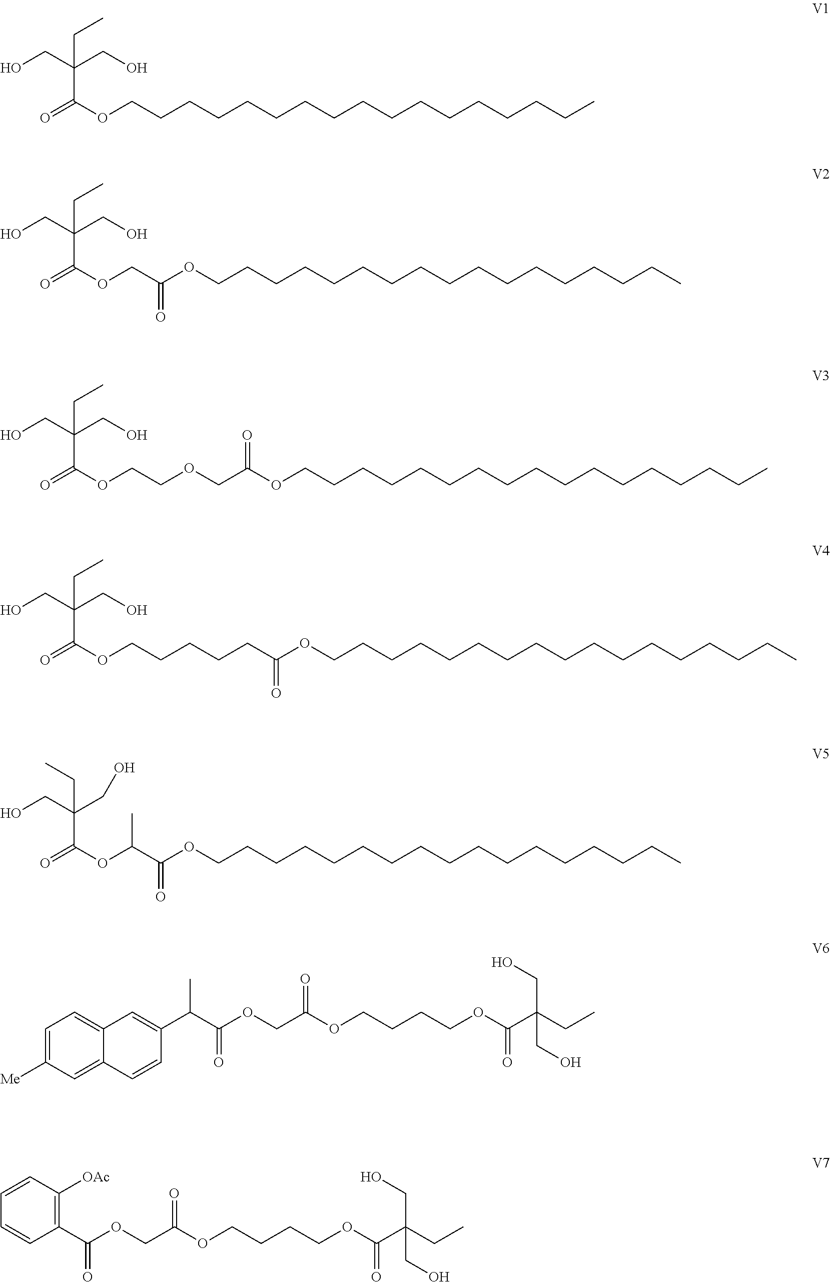

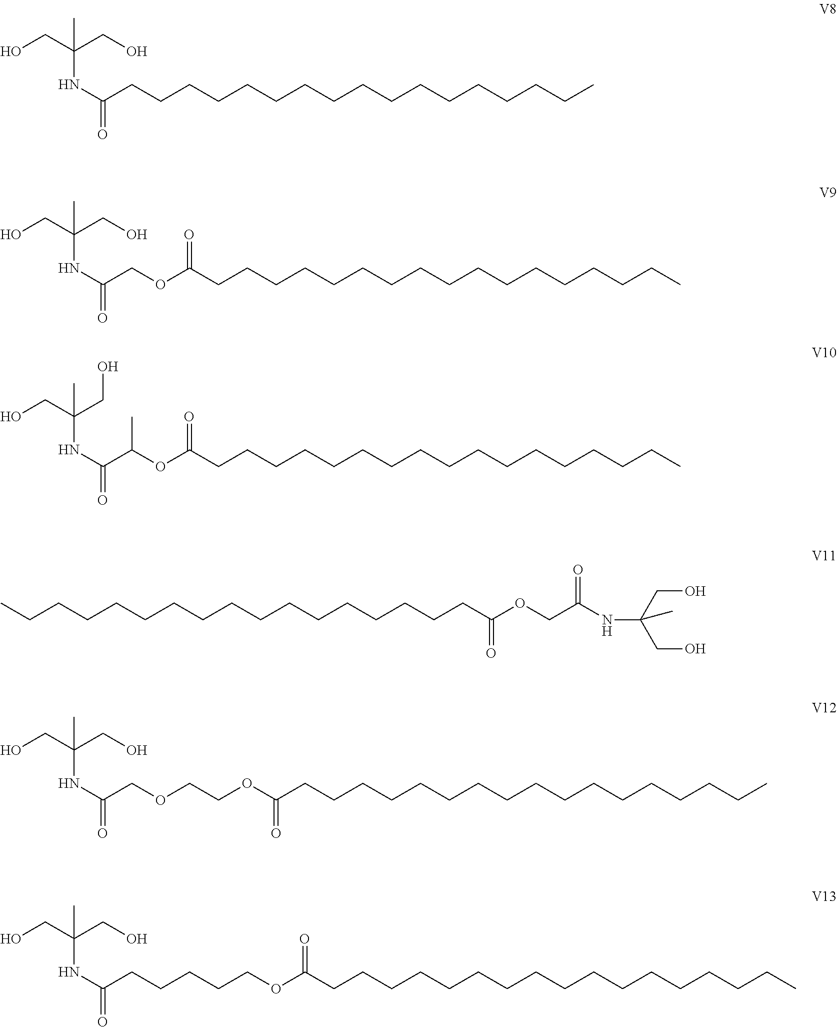

In other embodiments, branched absorbable polyurethanes of the present invention, such as in connection with their use as stents, stent coatings, films, highly porous artificial extracellular matrix or scaffold to accommodate mammalian cells and guide their growth and tissue regeneration, polyurethane foams and adhesion prevention barrier, may be derived from isocyanates of present invention and reacting them with chain extenders of Formula V described above and polyols of Formula VI including but not limited to the examples shown below (#s VI•1-VI•7):

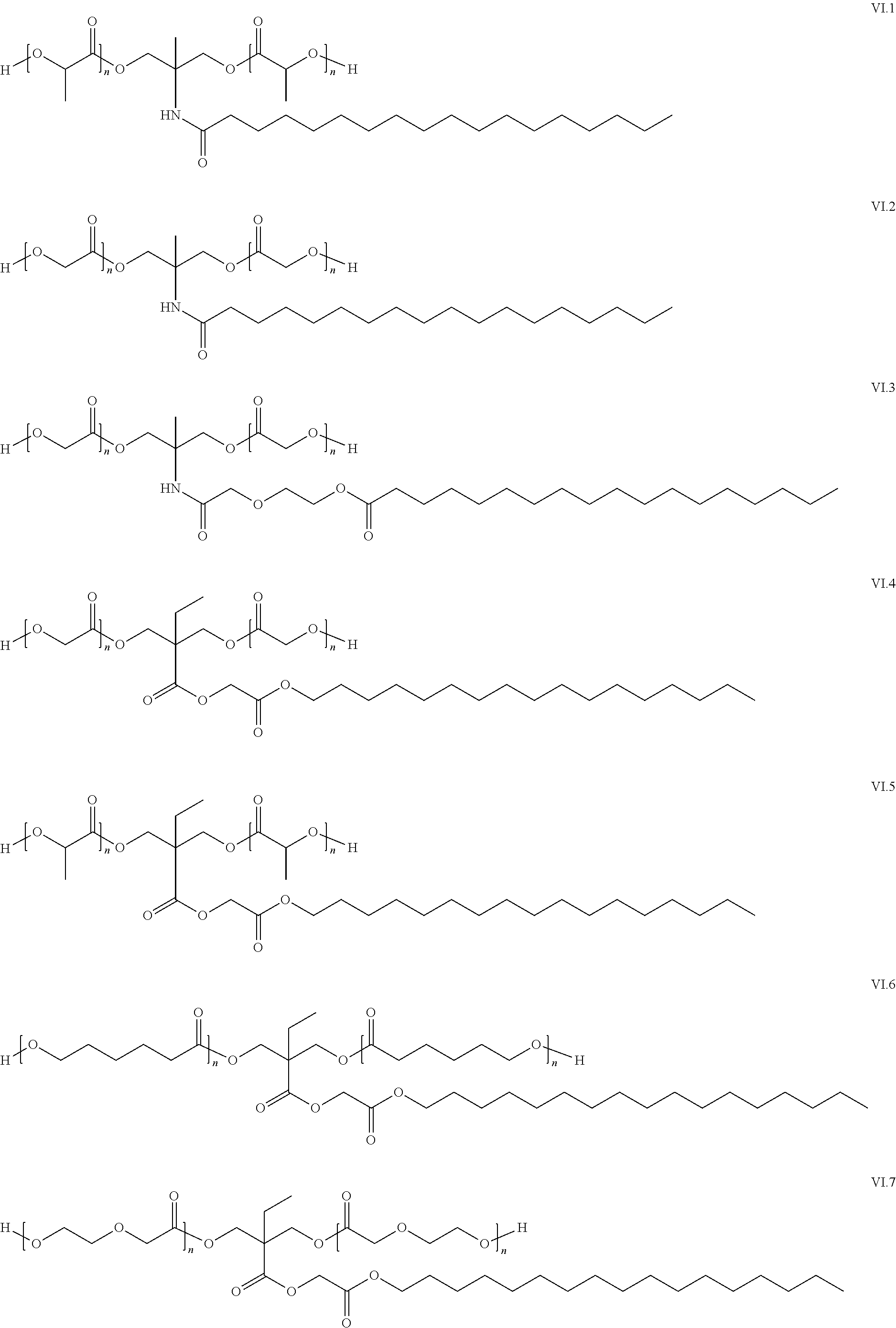

In other embodiments, branched absorbable polyurethanes of the present invention, such as in connection with their use as stents, stent coatings, films, adhesion prevention barrier, highly porous foam, reticulated foams, drug device combinations, medical device coatings, films, non-implantable medical device, scaffolds and polyurethane foams, may be derived from isocyanates described and claimed in U.S. Patent Application Publication Nos. 20060188547, 20090292029, European Patent Publication No. EP 1937182 and WO 2007030464 and reacting them with chain extenders of Formula V and polyols of Formula VI described herein.

In other embodiments, branched absorbable polyurethanes of the present invention may be derived from dicarboxylic acid based hydrolysable isocyanates with the general formula I and II, wherein if r is 1 to 2 and p, q is 0, e.g. structures#2 and 3, the isocyanates hydrolyze slowly. In contrast to this, if r>2 and p, q is 0, the rate of hydrolysis of isocyanates is very slow. Furthermore, isocyanates prepared from dicarboxylic acids functionalized on both sides with glycolic acid, lactic acid, caprolactone or p-dioxanone shown in general formula II generally have faster rate of hydrolysis as compared to isocyanates prepared from dicarboxylic acids functionalized on one side with glycolic acid, lactic acid, caprolactone or p-dioxanone shown in general formula I. Polyurethanes with tunable physical and biological properties can be prepared from the blend of isocyanates represented by general formulas I and II.

In another embodiment, branched absorbable polyurethanes of the present invention may be derived from safe and biocompatible amino acids. Polyurethanes resulting from these isocyanates are expected to be safe and biocompatible. Although all the naturally occurring and synthetic amino acids can be used as precursors for preparation of hydrolysable isocyanates in the present invention, however, examples in the present patent application are from Tyramine and Tyrosine. The structures of these isocyanates are represented by general formulas III and IV respectively.

In another embodiment, branched or linear absorbable polyurethanes of the present invention may also be derived from isocyanates based on cycloaliphatic amino acids such as aminocyclohexanecarboxylic acid as well as cycloaliphatic amino alcohols such as aminocyclohexanol. Polyurethanes from these isocyanates can be prepared according to the procedures described in U.S. Patent Application Publication Nos. 20060188547, 20090292029, European Patent Publication No. EP 1937182 and WO 2007030464. Polyurethanes resulting from cycloaliphatic amino acids as well as cycloaliphatic amino alcohols will find use in a variety of applications including biomedical applications wherein controlled hydrolytic degradation is desired.

In another embodiment branched absorbable polyesters can be prepared by reaction of branched chain extenders, such as of Formula V, and/or branched absorbable polyols, such as of Formula VI, with diacids including but not limited to oxalic acid, succinic acid, malonic acid, butanedioic acid, adipic acid, azelaic acid, sebacic acid, diglycolic acid, 3,6-dioxaoctanedioic acid, 3,6,9-trioxaundecanoic acid, functionalized oxaacids, polyethyleneglycol diacids of average molecular weight from 300 to 2000 and blends thereof.

In another embodiment branched absorbable polyesters will be prepared by reaction of blends of polyethylene glycols with branched chain extenders, such as of Formula V, and/or branched absorbable polyols, such as of Formula VI, and reacting with diacids including but not limited to oxalic acid, succinic acid, malonic acid, butanedioic acid, adipic acid, azelaic acid, sebacic acid, diglycolic acid, 3,6-dioxaoctanedioic acid, 3,6,9-trioxaundecanoic acid, functionalized oxaacids, polyethyleneglycol diacids of average molecular weight from 300 to 2000 and blends thereof.

In another embodiment of the present invention, branched absorbable polyesters of the present invention can be further polymerized with lactone monomers including but not limited to glycolide, lactide, caprolactone, p-dioxanone, TMC, δ-valerolactone, β-butyrolactone, morpholinedione, pivalolactone, ε-decalactone, 2,5-diketomorpholine and combinations thereof in order to control physical and biological properties.

It would be readily apparent to one of ordinary skill in the art once armed with the teachings in the present application that the isocyanates, chain extenders and polyols of the present invention may be reacted with a variety of reactants that are typically employed in the preparation of bioabsorbable and biocompatible polyurethanes and/or polyesters, preferably with tunable physical, mechanical properties and/or hydrolytic degradation profiles. It would also be apparent to the ordinarily skilled artisan that the terminal groups for given polyurethane, or polyester may be derivatized by further reacting the polyurethane and/or polyesters with additional derivatizing agents. Polyurethanes terminated with —NCO or hydroxyl groups can be prepared by varying the ratio of isocyanates:hydroxyl groups in the reaction mixture i.e. (isocyanates, chain extender and polyol). Polyurethanes with high molecular weights are formed when the ratio of isocyanates:hydroxyl group is 1. Furthermore, by varying the ratio of isocyanates:hydroxyl groups in the reaction mixture, polyurethanes with tunable physical and mechanical properties can be obtained. It would also be apparent to the ordinarily skilled artisan that the terminal groups for given polyurethane, or polyester may be derivatized by further reacting the polyurethane and/or polyesters with additional derivatizing agents.

In certain embodiments, the branched absorbable polyurethane and/or polyesters as described herein may be further polymerized with a lactone monomer, preferably selected from the group consisting of glycolide, lactide, caprolactone, p-dioxanone, TMC, δ-valerolactone, β-butyrolactone, morpholinedione, pivalolactone, ε-decalactone, 2,5-diketomorpholine and combinations thereof. In certain embodiments, the applications of the present invention comprise these further polymerized polyurethanes and/or polyesters.

In one form, the branched absorbable polyurethanes and polyesters described herein are biodegradable and in certain aspects biocompatible and suitable for use in medicine. Such polyurethanes, and/or polyesters combine the good mechanical properties of polyurethanes with the degradability of polyesters.

The branched absorbable polyurethane and/or polyesters herein is suitable for use in a wide variety of applications. Since the degradation products of the biocompatible polyurethanes and/or polyesters described herein are non-toxic, they are advantageously suitable for biomedical uses. For example, the properties of the polymer may be tunable, i.e., they may be made to degrade more slowly or more quickly by reducing or increasing respectively the number of ester linkages in the polymeric chain, and can thus be utilized for fabricating short-term or long-term implantable surgical materials.

The polyurethanes and/or polyesters may be formed into articles and formulations using any known technique, such as, for example, extrusion, molding and/or solvent casting, blending. The polyurethanes and/or polyesters may be used alone, blended with other absorbable compositions, or in combination with non-bioabsorbable components. A wide variety of articles or formulations may be manufactured from the polyurethanes and/or polyesters described herein. These include but are not limited to a stent, stent coating, wound film, covering or dressing, burn covering or dressing, surgical dressing, mesh, foam, highly porous foam, reticulated foam, drug device combination, medical device coating, tissue engineering scaffold, film, adhesion prevention barrier, implantable medical device, non-implantable medical device, controlled drug delivery device or system, anastomosis ring, or the like. These further include but are not limited to a clip or other fastener, suture, ligature, needle and suture combination, surgical clip, surgical staple, prosthesis (e.g., surgical), textile structure, coupling, tube, support, screw, pin, or the like. These further include but are not limited to a bone wax formulation, an adhesion prevention barrier, or the like.

In certain drug delivery systems, the systems comprise a polyurethane, and/or polyester in admixture with a biologically or pharmaceutically active agent. Non-limiting examples of polymeric carriers in such drug delivery systems and/or pharmaceutical compositions include self-supporting films, hollow tubes, beads, and/or gels. Other uses of the surgical article include their use as a scaffold for tissue engineering comprising a porous structure for the attachment and proliferation of cells, such as in vitro or in vivo. The polyurethanes and/or polyesters herein may also be used to fabricate degradable containers and packaging materials which can degrade in landfills in contrast to existing non-degradable materials which present environmental concerns.

The polymers of the present invention may be used as pharmaceutical carriers in a drug delivery matrix, i.e., a matrix for a biologically active agent. The matrix may be formed by mixing the polymer with a biologically active agent. The biologically active agent can be dispersed into the polymer solution for example during preparation of matrix or via melt blending. A vast variety of different biologically active agents may be used in conjunction with the polymers of the invention. In general, therapeutic agents administered via the pharmaceutical compositions of the invention include, without limitation: anti-infectives such as antibiotics and antiviral agents; analgesics and analgesic combinations; anorexics; anti-helmintics; anti-arthritics; anti-asthmatic agents; anticonvulsants; antidepressants; anti-diuretic agents; anti-diarrheals; anti-histamines; anti-inflammatory agents; anti-migraine preparations; anti-nauseants; anti-neoplastics; anti-parkinsonism drugs; anti-pruritics; anti-psychotics; anti-pyretics, anti-spasmodics; anti-cholinergics; sympathomimetics; xanthine derivatives; cardiovascular preparations including calcium channel blockers and beta-blockers such as pindolol and anti-arrhythmics; anti-hypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; para-sympatyholytics; psychostimulants; sedatives; and tranquilizers; and naturally derived or genetically engineered proteins, polysaccharides, glycoproteins or lipoproteins.

The drug delivery matrix may be administered in any suitable dosage form such as oral, parenteral, subcutaneously as an implant, vaginally or as a suppository. Matrix formulations containing polymers of the invention may be formulated by mixing one or more therapeutic agents with the polymer. The biologically active agent may be present as a liquid, a finely divided solid, or any other appropriate physical form. Typically, the matrix will include one or more additives, e.g., nontoxic auxiliary substances such as diluents, carriers, excipients, stabilizers or the like. However, the presence of such additives is optional. Other suitable additives may be formulated with the polymers of this invention and pharmaceutically active agent or compound. If water is to be used as an additive, it is preferably be added immediately before administration.

The amount of biologically active agent will be dependent upon the particular agent employed and medical condition being treated. Typically, the amount of drug represents about 0.001% to about 70%, more typically about 0.01% to about 50%, and most typically about 0.1% to about 20% by weight of the matrix.

The quantity and type of polymer incorporated into a parenteral dosage form will vary depending on release profile desired and the amount of drug employed. The product may contain blends of polymers of this invention to provide the desired release profile or consistency to a given formulation.

The polymers of this invention, upon contact with body fluids including blood or the like, undergo gradual degradation (mainly through hydrolysis) with concomitant release of the dispersed drug for a sustained or extended period (as compared to the release from an isotonic saline solution). This may result in prolonged delivery (over about one to about 2,000 hours, preferably about two to about 800 hours) of effective amounts (including, for example, about 0.0001 mg/kg/hour to about 10 mg/kg/hour) of the drug. This dosage form may be administered as necessary depending on the subject being treated, the severity of the affliction, the judgment of the prescribing physician, and the like.

Individual formulations of drugs and polymers of this invention may be tested in appropriate in vitro and in vivo models to achieve the desired drug release profiles. For example, a drug may be formulated with a polymer of this invention and administered to an animal (e.g., orally). The drug release profile may be monitored by appropriate means, such as by taking blood samples at specific times and assaying the samples for drug concentration. Following this or similar procedures, those skilled in the art may formulate a variety of formulations.

The branched absorbable polyurethane material of the present invention are believed to be useful for use as a tissue engineering scaffold, i.e., as a structure for the growth or regeneration of tissue. Polyurethanes may lend themselves to such uses since the enzyme-catalyzed degradation may in some cases work concurrently with the migration or growth of cells into the material, while desirably degrading in the process into its substantially non-toxic constituents. It is also possible, in some cases, that cells migrating into or located adjacent the matrix may themselves exude proteolytic enzymes that will also mediate hydrolytic cleavage.

Such tissue engineering scaffolds may have applications in the regeneration of skin and other organs, bone, cartilage, ligaments, tendons, bladder and other tissue. The polyurethane material may also be useful in the production of sutures, which require good mechanical strength, and drug release matrices, in view of their need for non-toxic degradability. The polyurethane material may also be useful for other non-biomedical applications, where degradability into substantially non-toxic constituents is an asset. The polyurethane material lends itself to sterilization by such techniques as gamma radiation and ethylene oxide treatments.

Fibers made from the present polyurethanes and/or polyesters can be knitted or woven with other fibers, either absorbable or non-absorbable to form meshes or fabrics. Compositions including these polyurethanes and/or polyesters may also be used as absorbable coating for surgical devices.

In another aspect, the compositions containing the polyurethanes and/or polyesters described herein may be used to make reinforced composites. Thus, for example, the polyurethane and/or polyester composition may form the matrix of the composite and may be reinforced with bioabsorbable or non-bioabsorbable fibers or particles. Alternatively, a matrix of any absorbable or non-bioabsorbable polymer composition may be reinforced with fibers or particulate material made from compositions containing the polyurethanes and/or polyesters described herein.

In an alternative embodiment, the branched absorbable polyurethanes, and/or polyesters described herein may be admixed with a filler. The filler may be in a particulate form, such as granulates and staple fibers. While any known filler may be used, hydroxyapatite, tricalcium phosphate, bioglass or other bioceramics are the exemplary fillers. For example, from about 10 grams to about 400 grams of filler are mixed with about 100 grams of polymer. The filled, cross-linked polymers are useful, for example, as a molding composition.

It is further contemplated that one or more medico-surgically useful substances (biologically active agents) may be incorporated into compositions containing the branched absorbable polyurethanes and/or polyesters described herein. Examples of such biologically active agents include, for example, those which accelerate or beneficially modify the healing process when particles are applied to a surgical repair site. For example, articles made from compositions containing the present polyurethanes and/or polyesters may carry a therapeutic agent which will be deposited at the repair site. The biologically active agent may be chosen for its antimicrobial properties, capability for promoting repair or reconstruction and/or new tissue growth. Antimicrobial agents such as broad spectrum antibiotic, for example, gentamycin sulfate, erythromycin, or derivatized glycopeptides which are slowly released into the tissue may be applied in this manner to aid in combating clinical and sub-clinical infections in a tissue repair site. To promote repair and/or tissue growth, one or several growth promoting factors may be introduced into the articles, e.g., fibroblast growth factor, bone growth factor, epidermal growth factor, platelet derived growth factor, macrophage derived growth factor, alveolar derived growth factor, monocyte derived growth factor, magainin, and the like. Some therapeutic indications are: glycerol with tissue or kidney plasminogen activator to cause thrombosis, superoxide dismutase to scavenge tissue damaging free radicals, tumor necrosis factor for cancer therapy or colony stimulating factor and interferon, interleukin-2 or other lymphokine to enhance the immune system.

It is contemplated that it may be desirable to dye articles made from compositions containing the present branched absorbable polyurethanes and/or polyesters in order to increase visibility of the article in the surgical field. Dyes, such as those known to be suitable for incorporation in sutures, may be used. Such dyes include but are not limited to carbon black, bone black, D&C Green No. 6, and D&C Violet No. 2 as described in the handbook of U.S. Colorants for Food, Drugs and Cosmetics by Daniel M. Marrion (1979), the disclosures of which are hereby incorporated herein by reference, in their entireties. Preferably, articles in accordance with this disclosure may be dyed by adding up to about a few percent and preferably about 0.2% dye to the resin composition prior to extrusion.

Biologically active hydroxy compounds that can be used as a pendant group of the present invention include acenocoumarol, acetarsol, actinoquinol, adrenalone, alibendol, amodiaquine, anethole, balsalazide, bamethan, benserazide, bentiromide, benzarone, benzquinamide, bevantolol, bifluranol, buclosamide, bupheniode, chlorotrianisene, chloroxylenol, cianidanol, cinepazide, cinitapride, cinepazide, cinmetacin, clebopride, clemastine, clioquinol, cyclovalone, cynarine, denopamine, dextroythyroxine, diacerein, dichlorophen, dienestrol, diethylstilbestrol, diflunisal, diiodohydroxyquinoline, dilazep, dilevalol, dimestrol, dimoxyline, diosmin, dithranol, dobutamine, donepezil, dopamine, dopexamine, doxazosin, entacapone, epanolol, epimestrol, epinephrine, estradiol valerate, estriol, estriol succinate, estrone, etamivan, etamsylate, ethaverine, ethoxzolamide, ethyl biscoumacetate, etilefrine, etiroxate, exalamide, exifone, fendosal, fenoldopam mesilate, fenoterol, fenoxedil, fenticlor, flopropione, floredil, fluorescein, folescutol, formoterol, gallopamil, gentistic acid, glaziovine, glibenclamide, glucametacin, guajacol, halquinol, hexachlorophene, hexestrol, hexobendine, hexoprenaline, hexylresorcinol, hydroxyethyl salicylate, hydroxystilbamidine isethionate, hymecromone, ifenprodil, indomethacin, ipriflavone, isoetarine, isoprenaline, isoxsuprine, itopride hydrochloride, ketobemidone, khellin, labetalol, lactylphenetidin, levodopa. levomepromazine, levorphanol, levothyroxine, mebeverine, medrylamine, mefexamide, mepacrine, mesalazine, mestranol, metaraminol, methocarbamol, methoxamine, methoxsalen, methyldopa, midodrine, mitoxantrone, morclofone, nabumetone, naproxen, nitroxoline, norfenefrine, normolaxol, octopamine, omeprazole, orciprenaline, oxilofrine, oxitriptan, oxyfedrine, oxypertine, oxyphenbutazone, oxyphenisatin acetate, oxyquinoline, papaverine, paracetanol, parethoxycaine, phenacaine, phenacetin, phenazocine, phenolphthalein, phenprocoumon, phentolamine, phloedrine, picotamide, pimobendan, prenalterol, primaquine, progabide, propanidid, protokylol, proxymetacaine, raloxifene hydrochloride, repaglinide, reproterol, rimiterol, ritodrine, salacetamide, salazosulfapyridine, salbutamol, salicylamide, salicylic acid, salmeterol, salsalate, sildenafil, silibinin, sulmetozin, tamsulosin, terazosin, terbutaline, tetroxoprim, theodrenaline, tioclomarol, tioxolone, alpha-tocopherol (vitamin E), tofisopam, tolcapone, tolterodine, tranilast, tretoquinol, triclosan, trimazosin, trimetazidine, trimethobenzamide, trimethoprim, trimetozine, trimetrexate glucuronate, troxipide, verapamil, vesnarinone, vetrabutine, viloxazine, warfarin, xamoterol.

Other biologically active phenolics that can be used include acacetin, 4-acetamido-2-methyl-1-naphthol, acetaminophen, albuterol, allenolic acid, aloe emodin, aloin, β-amino-4-hydroxy-3,5-di-iodohydrocinnamic acid, N-(5-amino-2-hydroxyphenyl)-benzeneacetamide, 4-amino-1-naphthol, 3-aminosalicylic acid, 4-aminosalicylic acid, anacardic acid, p-anol, anthragallol, anthralin, anthranol, anthrarobin, anthrarobin, apigenin, apiin, apocynin, aspidinol, aspirin, baptigenin, benzestrol, benzoresorcinol, bisphenol a, bisphenol b, butylated hydroxylanisole, butylated hydroxytoluene, capobenic acid, trans-1-(3′-carboxy-4′-hydroxyphenyl)-2-(2″,5″-dihydroxyphenyl)ethane, catechin, chlorogenic acid, m-chlorophenol, 5-chloro-8-quinolinol, chloroxylenol, chlorquinaldol, chromo-nar, chrysin, cinametic acid, clorophene, coniferyl alcohol, p-coumaric acid, coumes-trol, coumetarol, daphnetin, datiscetin, deoxyepinephrine, 3,5-diiodothyronine, 3,5-di-iodotyrosine, dimethophrine, diosmetin, diresorcinol, disoprofol, dopa, dopamine, drosophilin a, efloxate, ellagic acid, embelin, Equol, eriodictyol, esculetin, esculin, ethylnorepinephrine, ethyl vanillin, eugenol, eupatorin, fenadiazole, ferulic acid, fisetin, 3-fluoro-4-hydroxyphenylacetic acid, fraxetin, fustin, galangin, gallacetophe-none, gallic acid, gardenins, genistein, gentisyl alcohol, gepefrine, geranylhydroqui-none, [6]-gingerol, gossypol, guaiacol, guaifenesin, harmalol, hematoxylin, hinderin, homoeriodictyol, homogentisic acid, homovanillic acid, hydroxyamphetamine, 2-hyd-roxy-5-(2,5-dihydroxybenzylamino)-2-hydroxybenzoic acid, 4-hydroxy-3-methoxy-mandelic acid, n-(p-hydroxyphenyl)glycine, hydroxyprocaine, 8-hydroxyquinoline, hypericin, irigenin, isoproterenol, isoquercitrin, isothebaine, kaempferol, liothyronine, luteolin, mangostin, 5,5′-methylenedisalicylic acid, n-methylepinephrine, metyrosine, morin, mycophenolic acid, myricetin, naringenin, nylidrin, orcinol, osalmid, osthole, oxantel, paroxypropione, pentachlorophenol, 3-pentadecylcatechol, p-pentyloxy-phenol, phloretin, phloroglucinol, pinosylvine, plumbagin, pyrocatechol, pyrogallol, quercetagetin, quercetin, resacetophenone, rhamnetin, rhein, sakuranetin, salicyl alcohol, salicylanilide, 4-salicyloylmorpholine, salsalate, scopoletin, scutellarein, serotonin, (3,4,5-trihydroxyphenyl) methylenepropanedinitrile, thymol, thyropropic acid, thyroxine, tiratricol, tyrosine, vanillic acid, and vanillin.

Further biologically active carboxylic acid and/or amine compounds that can be used as a pendant group of the present invention include Acemetacin, Aceclofenac, Acediasulfone, Adipiodone, Alminoprofen, Amisulpride, Amlexanox, Amodiaquine, Amosulalol, Amoxicillin, Amsacrine, Anileridine, Azacyclonol, Baccofen, Balsalazide sodium, Bentiromide, Benzocaine, Bromopride, Bumetanide, Carprofen, Carvedilol, Carzenide, Cefprozil, Cinitapride, Cinmetacin, Clebopride, Clenbuterol, Clometacin, Cromoglicic acid, Diclofenac, Diflunisal, Eprosartan, Ethoxzolamide, Fendosal, Flufenamic acid, Furosemide, Ibuprofen, Indometacin, Iobenzamic acid, Iocarmic acid, Iocetamic acid, Iodoxamic acid, Ioglycamic acid, Iophenoic acid, Iotroxic acid, Mefenamic acid, Nadoxolol, Naproxen, Nedocromil, D-Norpseudoephedrine, paracetamol Repaglinide, Salazosulfapyridine, Salicylic Acid, Salsalate and Sarpogrelate.

Examples of biologically active dihydroxy compounds that can be used to as a pendant group of present invention include Adrenalone, Alfuzosin, Alibendol, Amrubicin, Apomorphine, Bamethan, Benzquinamide, Bevantolol, Bifluranol, Bisacodyl, Brodimoprim, Bunazosin, Bupheniode, Carbidopa, Carbuterol, Cyclofenil, Cyclovalone, Daunorubicin, Dichlorophen, Dienestrol, Diethylstilbestrol, Dimestrol, Dithranol, Donepezil, Doxefazepam, Doxorubicin, Entacapone, Epinepheine, Epirubicin, Esomeprazole, Etamivan, Etamsylate, Etilefrine, Ezetimibe, Fenticlor, Fluorescein, Folescutol, Formoterol, Gefitinib, Hexestrol, Hexylresorcinol, Hydroxyethyl salicylate, Ifenprodil, Isoetarine, Isoxsuprine, Itopride. HCl, Khellin, Labetalol, Mitoxantrone, Morclofone, Moxaverine, Normolaxol, Omeprazole, Oxilofrine, Oxepertine, Phenacaine, Phenolphthalein, Prazosin, Tolcapone, Vesnarinone, and Vetradutine.

Examples of biologically active diamino compounds that can be used as a pendant group of present invention include Amisulpride, Amodiaquine, Amosul-alol, Amoxicillin, Amsacrine, Azacyclonol, Bromopride, Carvedilol, Cefprozil, Cinitapride, Clebopride, Clenbuterol, Ethoxzolamide, Nadoxolol, and D-Norpseudoephedrine.

Examples of biologically active hydroxy/amino compounds that can be used as a pendant group include Amisulpride, Amodiaquine, Amosulalol, Amoxicillin, Amsacrine, Azacyclonol, Bromopride, Carvedilol, Cefprozil, Cinitapride, Clebopride, Clenbuterol, Ethoxzolamide, Nadoxolol, D-Norpseudo-ephedrine, and paracetamol.

Examples of biologically active dicarboxylic acid compounds that can be used as a pendant group of present invention include Adipiodone, Cromoglicic acid, Eprosartan, Iocarmic acid, Iodoxamic acid, Ioglycamic acid, Iotroxic acid, Nedocromil.

Examples of biologically active hydroxy/carboxylic acid compounds that can be used as a pendant group of present invention include Acemetacin, Bentiromide, Cinmetacin, Clometacin, Diflunisal, Fendosal, Indometacin, Iophenoic acid, Naproxen, Repaglinide, Salazosulfapyridine, Salicylic Acid, Salsalate, and Sarpogrelate.

Examples of biologically active hydroxyl-acids for use as the pendant group include 4-hydroxycinnamic acid, caffeic acid, chlorogenic acid, ferulic acid, sinapic acid, vanillic acid, Acemetacin, Bentiromide, Cinmetacin, Clometacin, Diflunisal, Fendosal, Indometacin, Iophenoic acid, Naproxen, Repaglinide, Salazosulfapyridine, Salicylic Acid, Salsalate, and Sarpogrelate.

Examples of useful biologically active amino/carboxylic acid compounds that can be used as a pendant group of present invention include Aceclofenac, Acediasulfone, Alminoprofen, Amlexanox, Anileridine, Baccofen, Balsalazide sodium, Benzocaine, Bumetanide, Carprofen, Carzenide, Diclofenac, Flufenamic acid, Furosemide, Iobenzamic acid, Iocetamic acid, and Mefenamic acid.

Examples of biologically active diamino compounds useful in the present invention include Amisulpride, Amodiaquine, Amosulalol, Amoxicillin, Amsacrine, Azacyclonol, Bromopride, Carvedilol, Cefprozil, Cinitapride, Clebopride, Clenbuterol, Ethoxzolamide, Nadoxolol, D-Norpseudoephedrine, amino acids (L-lysine), and natural products.

Examples of naturally occurring biologically active phenolics include bergaptol, caffeic acid, capsaicin, coumarin, daidzein, 2,5-dihydroxy-benzoic acid, ferulic acid, flavonoids, glycitein (isoflavone), 4-hydroxycinnamic acid, 4-hydroxy-coumarin, isopimpinellin, resveratrol, synapic acid, vanillic acid, vanillin, chalcones, soybean flavonoids and derivatives thereof.

Capsaicin is a biologically active phenolic that is the active component of cayenne pepper. The capsaicin is an amide of vanillylamine and C₈ to C₁₃ branched fatty acids. Topical application of capsaicin stimulates and blocks small pain fibers by depleting them of the neurotransmitter substance P that mediates pain impulses. A cream made from 0.025%-0.075% capsaicin applied 4× daily may help peripheral neuropathic pain, post-herpetic neuralgia, trigeminal neuralgia, psoriasis and fibromyalgia. It is also useful for diabetic neuropathy, cluster headaches, earache, osteo- and rheumatoid arthritis. Capsaicin is a powerful pain reliever.

Naproxen, paracetamol, acetaminophen and acetylsalicylic acid are biologically active phenolics that belong to the class of drugs called non-steroidal anti-inflammatory drugs or NSAIDs. The NSAIDs provide relief by blocking the action of prostaglandins, which are hormone-like substances that contribute to pain, inflammation, fever and muscle cramps. Phenolic moieties, synthetic and naturally occurring, are part of many drugs.

The compounds employed in the methods of the present invention may be prepared in a number of ways well known to those skilled in the art. The compounds may be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

The following examples are included to further illustrate the invention and are not to be considered as limiting the invention anyway. Melting points were measured for all products by using a Polmon (MP 96) melting point apparatus. For all the products, NMR was run using a Varian 200 MHz and tetramethylsilane as an internal standard.

### EXAMPLES

### Example 1

### Synthesis of Succinic acid bis-(4-nitro-phenyl) ester

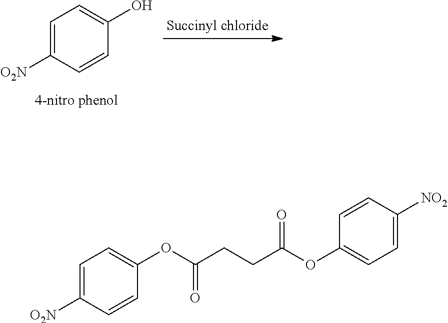 [SMILES: O=[N+]([O-])c1ccc(O)cc1 ; O=C(CCC(=O)Oc1ccc([N+](=O)[O-])cc1)Oc1ccc([N+](=O)[O-])cc1]

To a solution of 4-nitro phenol (273 grams, 1.962 mol) and pyridine (190 ml, 2.349 mol) in dichloromethane (1500 ml) at 0° C. was added dropwise succinyl chloride (145 grams, 0.935 mol). The reaction mixture was left for further stirring at room temperature for 16 hours. The reaction mixture was poured into cold water. The separated solid was filtered, dried and recrystallised from a mixture of dimethyl formamide and methanol (3:5) to yield 185 grams of pure Succinic acid bis-(4-nitro-phenyl) ester as white powder with a melting point of 184-186° C. The compound was characterized by ^{I}H NMR (DMSO-d₆) δ 3.05 (s, 2H, CH₂), 7.45 (d, 2H, Ar), 8.33 (d, 2H, Ar).

### Example 2

### Synthesis of Succinic acid Succinic acid bis-(4-amino-phenyl) ester

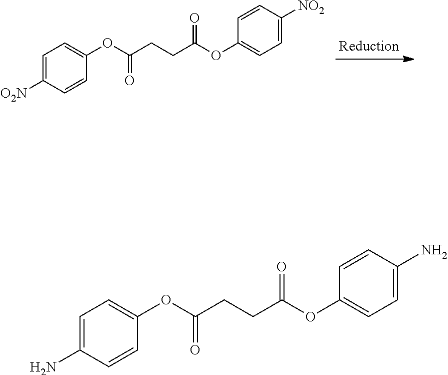 [SMILES: O=C(CCC(=O)Oc1ccc([N+](=O)[O-])cc1)Oc1ccc([N+](=O)[O-])cc1 ; Nc1ccc(OC(=O)CCC(=O)Oc2ccc(N)cc2)cc1]

To a solution of Succinic acid bis-(4-nitro-phenyl) ester (35 grams, 97.22 mmoles) in Dimethyl formamide (150 ml) in a pressure vessel was added 10% Palladium carbon (4 grams, 50% wet). The reaction mixture was stirred under an atmosphere of hydrogen (4 Kg) for 2 hours. The catalyst was removed by filtration, and crude Succinic acid bis-(4-amino-phenyl) ester was precipitated by adding methanol. It was filtered and dried to yield 22 grams of pure Succinic acid bis-(4-amino-phenyl) ester as white powder with a melting point of 182-184° C. The product was characterized by ^{I}H NMR (DMSO-d₆) δ 2.82 (s, 2H, CH₂), 5.02 (s, 2H, NH₂), 6.52 (d, 2H, Ar), 6.82 (d, 2H, Ar). Hydrolysis of 0.5 grams of Succinic acid bis-(4-amino-phenyl) ester in a 50 ml buffer of pH 9 showed 10% hydrolysis in 72 hours.

### Example 3

### Synthesis of Succinic acid bis-(4-isocyanato-phenyl) ester

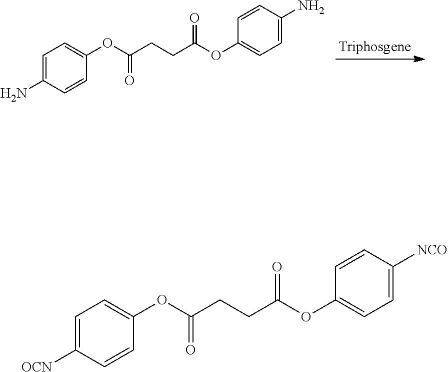

Into a stirring solution of Succinic acid bis-(4-amino-phenyl) ester (100 grams, 333 mmoles) in dry 1,4-Dioxane (2400 ml) maintained at 100° C. under Nitrogen atmosphere was added a solution of Triphosgene (198 grams, 667.79 mmoles) in 1,4-Dioxane. The solution was heated to reflux for 3 hours. The solvent was distilled off at atmospheric pressure until the volume of the reaction mixture was reduced to approximately one third. Fresh dry Dioxane (300 ml) was added and distilled off the solvents under vacuum. The residue was dissolved in Toluene (1000 ml) and charcoal (30 grams) was added, filtered hot, distilled off 80% Toluene and the residue was precipitated by adding dry Hexane (2000 ml). The precipitate was filtered off the separated to yield 93 grams of pure Succinic acid bis-(4-isocyanato-phenyl) ester as a white powder with a melting point of 102-104° C. The product was characterized by ^{I}H NMR (CDCl₃) δ 3.00 (s, 2H, CH₂), 7.04-7.11 (m, 4H, Ar). The isocyanate was converted into a urethane with a melting point of 193-195° C. in order to determine rate of hydrolysis of isocyanate. Hydrolysis of 0.1 grams of diurethane in a 10 ml buffer of pH 9 at 50° C. showed 10% hydrolysis in 72 hours.

### Example 4

### Synthesis of polyurethane from Succinic acid bis-(4-isocyanato-phenyl) ester

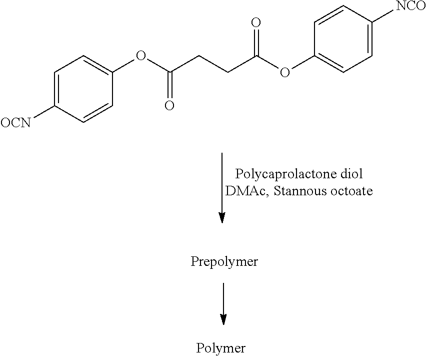

Into a clean flame dried 250 ml 4 neck round bottom flask maintained under nitrogen atmosphere at 28° C. in a oil bath was added diisocyanate (6.6 grams) dissolved in DMAc (25 ml). Into this stirring solution was added polycaprolactone diol (13 grams) and the solution was left for stirring at room temperature for another 30 minutes when the temperature of the bath was raised to 60° C. 3 drops of 10% stannous octoate solution was added. Additional quantities of DMAc were added followed by stirring of the reaction mixture at 60° C. for additional 30 minutes. The prepolymer formed turned the solution viscous.

To this solution of prepolymer was added 1,4-Butanediol (500 mg) dissolved in dimethylacetamide (5 ml). To this reaction mixture was added 5 drops of 10% stannous octoate solution. The reaction mixture was left for stirring at 60° C. for 1 hour. A small fraction of the reaction mixture was poured onto a petri dish to generate film of good strength.

### Example 5

### Synthesis of polyurethane from Succinic acid bis-(4-isocyanato-phenyl) ester

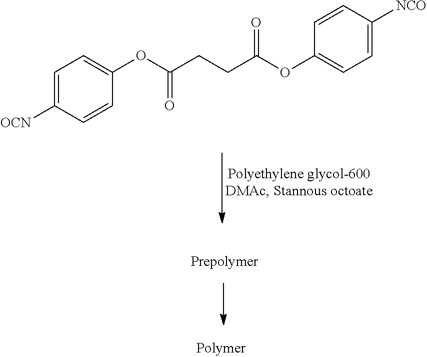

Into a clean flame dried 250 ml, 4 neck round bottom flask maintained under nitrogen atmosphere at 28° C. in an oil bath was added diisocyanate (7.3 grams) dissolved in DMAc (20 ml). Into this stirring solution was added polyethylene glycol with a average molecular weight of 600 (5 grams) and the solution was left for stirring at room temperature for another 30 minutes when the temperature of the bath was raised to 60° C. 3 drops of 10% stannous octoate solution was added. Additional quantities of DMAc were added followed by stirring of the reaction mixture at 60° C. for additional 3 hours. The prepolymer formed turned the solution viscous.

To this solution of prepolymer was added 1,4-Butanediol (550 mg) dissolved in dimethylacetamide (5 ml). To this reaction mixture was added 5 drops of 10% stannous octoate solution. The reaction mixture was left for stirring at 60° C. for 1 hour. A small fraction of the reaction mixture was poured onto a petri dish to generate film of good strength.

### Example 6

### Synthesis of Succinic Acid Dibenzyloxy Carbonyl Methyl Ester

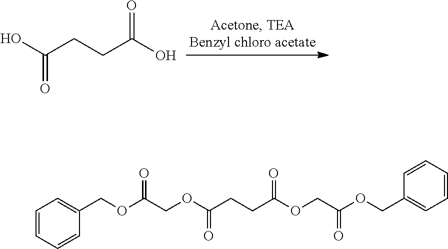 [SMILES: O=C(O)CCC(=O)O ; O=C(CCC(=O)OCC(=O)OCc1ccccc1)OCC(=O)OCc1ccccc1]

To a mixture of Succinic acid (250 grams, 2.118 mol), Triethyl amine (738 ml, 5.294 mol) in Acetone (2500 ml) at room temperature was added Benzyl chloro acetate (783 grams, 4.241 mol) dropwise and stirred at room temperature for 16 hours. The reaction mixture poured onto cold water, filtered separated solid, washed with chilled water, dried and purified by recrystallizing from Chloroform:Hexane (1:2) to yield pure Succinic acid dibenzyloxy carbonyl methyl ester (660 grams) as white powder with a melting point of 118-120° C. The product was characterized by ^{I}H NMR (CDCl₃) δ 2.80 (s, 2H, CH₂), 4.70 (s, 2H, CH₂), 5.20 (s, 2H, CH₂), 7.35 (m, 5H, Ar).

### Example 7

### Synthesis of Succinic Acid Dicarboxymethyl Ester

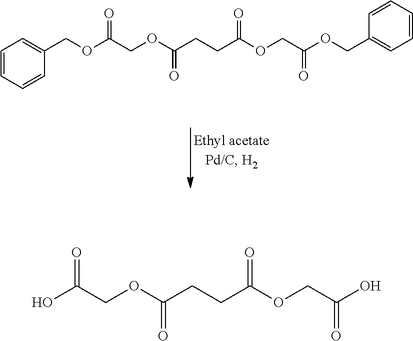 [SMILES: O=C(CCC(=O)OCC(=O)OCc1ccccc1)OCC(=O)OCc1ccccc1 ; O=C(O)COC(=O)CCC(=O)OCC(=O)O]

Succinic acid dibenzyloxy carbonyl methyl ester (660 grams, 1.594 mol) was dissolved in Ethyl acetate (2200 ml) in a pressure vessel, 50% wet Palladium on carbon (5%, 40 grams) added and the mixture stirred under an atmosphere of hydrogen (6 Kg) for 8 hours. The catalyst was removed by filtration and distilled off Ethyl acetate under vacuum to get the residue, which was precipitated by adding a mixture of Ethyl acetate and Hexane (1:1) to yield pure Succinic acid carboxymethyl ester (220 grams) as white powder with a melting point of 159-161° C. The product was characterized by ^{I}H NMR (DMSO-d₆) δ 2.70 (s, 2H, CH₂), 4.60 (s, 2H, CH₂).

### Example 8

### Synthesis of Succinic acid bis-(4-nitro-phenoxycarbonylmethyl) ester

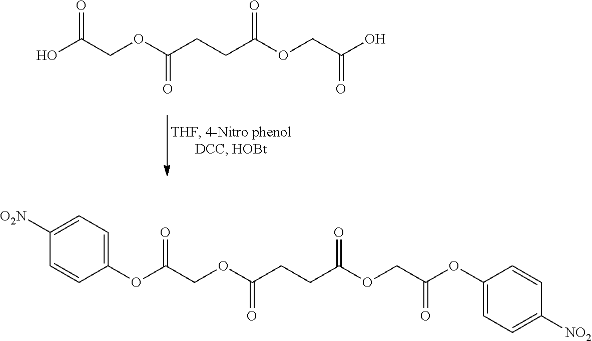 [SMILES: O=C(O)COC(=O)CCC(=O)OCC(=O)O ; O=C(CCC(=O)OCC(=O)Oc1ccc([N+](=O)[O-])cc1)OCC(=O)Oc1ccc([N+](=O)[O-])cc1]

To a mixture of 4-Nitro phenol (142.6 grams, 862.26 mmol), Succinic acid carboxymethyl ester (100 grams, 561.79 mmol), Hydroxy Benzotriazole (34.5 grams, 255.17 mmol) in Tetrahydrofuran (2000 ml) under Nitrogen at 0° C., was added dropwise a solution of DCC (233 grams, 1.129 mol) in Tetrahydrofuran (300 ml) and allowed to slowly come to room temperature and further stirred for 26 hours. The reaction mixture was filtered and washed with hot Tetrahydrofuran, distilled off the solvent under vacuum. The residue dissolved in Chloroform (2000 ml), washed with 5% Sodium bicarbonate (3000 ml), 1N HCl (3000 ml), water (1000 ml), dried over Sodium sulphate, distilled under reduced pressure, residue precipitated by adding Methanol, filtered, dried to give crude Octadecanoic acid (2,2,5-trimethyl-[1,3]dioxan-5-ylcarbamoyl)-methyl ester, which was purified by recrystallising in a mixture of Chloroform and Methanol (4:6) to yield 93 grams as white powder with a melting point of 125-128° C. The product was characterized by ^{I}H NMR (CDCl₃) δ 2.80 (s, 2H, CH₂), 4.90 (s, 2H, CH₂), 7.30 (d, 2H, Ar), 8.30 (d, 2H, Ar).

### Example 9

### Synthesis of Succinic acid bis-(4-amino-phenoxy carbonyl methyl) ester

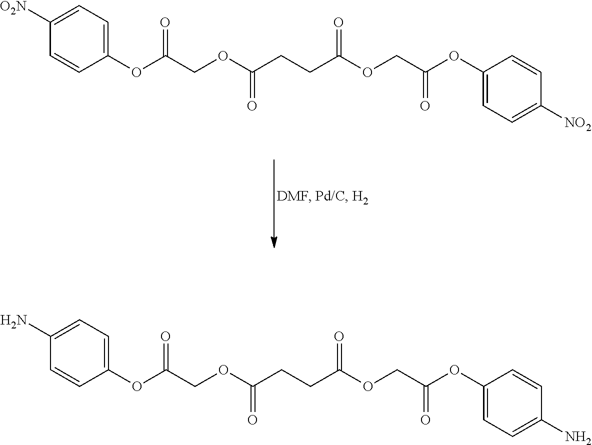 [SMILES: O=C(CCC(=O)OCC(=O)Oc1ccc([N+](=O)[O-])cc1)OCC(=O)Oc1ccc([N+](=O)[O-])cc1 ; Nc1ccc(OC(=O)COC(=O)CCC(=O)OCC(=O)Oc2ccc(N)cc2)cc1]

Succinic acid bis-(4-nitro-phenoxycarbonylmethyl) ester (20 grams, 42.02 mmoles) was dissolved in Dimethyl formamide (150 ml) in a Pressure vessel, 5% Palladium carbon (3 grams, 50% wet) added and the mixture stirred under an atmosphere of Hydrogen (5 Kg) for 1 hour. The catalyst was removed by filtration, and Diamine was precipitated by adding water, filtered, dried and slurried with hot ethyl acetate to yield pure Succinic acid bis-(4-amino-phenoxy carbonyl methyl) ester (18 grams) as brown powder with a melting point of 130-132° C. The product was characterized by ^{I}H NMR (DMSO-d₆) δ 2.75 (s, 2H, CH₂), 4.80 (s, 2H, CH₂), 5.10 (bs, 2H, NH₂), 6.50 (d, 2H, Ar), 6.75 (d, 2H, Ar). Hydrolysis of 0.5 grams of diamine in a 50 ml buffer of pH 9 at 50° C. showed complete hydrolysis in 90 minutes.

### Example 10

### Synthesis of Succinic acid bis-(4-isocyanato-phenoxy carbonyl methyl) ester

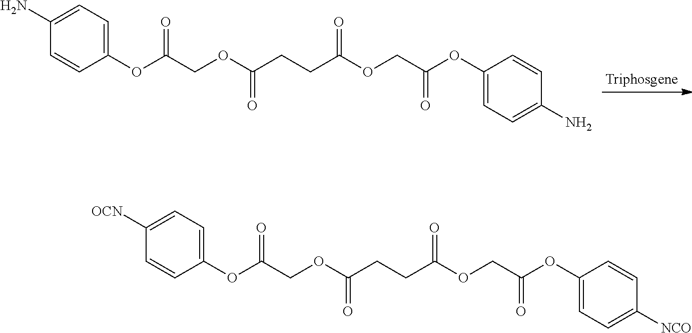

To a solution of Succinic acid bis-(4-amino-phenoxy carbonyl methyl) ester (25 grams, 60.09 mmoles) in dry 1,4-Dioxane (500 ml) under N₂ atmosphere was heated to 85° C. and added a solution of Triphosgene (35 grams, 117.94 mmoles) in 1,4-Dioxane (100 ml) in one lot and heated to 100° C. temperature for 2 hours. The condenser was then arranged for distillation and solvent removed by distillation at atmospheric pressure until the volume of the reaction mixture was reduced to approximately one third. Fresh dry Dioxane (75 ml) was added and distilled off the solvents under vacuum and this process of repeated distillation was continued for two more times by adding the same quantity of 1,4-Dioxane. The residue was dissolved in Toluene (75 ml), added charcoal (5 grams), filtered hot, precipitated the DiNCO by adding Hexane (200 ml), filtered redissloved in Toluene (150 ml) by heating to 100° C., filtered and precipitated by adding to cold Hexane (450 ml), filtered, dried and packed in tight container to yield 13 grams of pure [2-(4-Isocyanato phenoxy carbonyl methoxy)-ethoxy]-acetic acid 4-isocyanato-phenyl ester as a white powder with a melting point of 133-136° C. The product was characterized by ^{I}H NMR (CDCl₃) δ 2.80 (s, 2H, CH₂), 4.85 (s, 2H, CH₂), 7.10 (m, 4H, Ar). The isocyanate was converted into a urethane with a melting point of 149-151° C. in order to determine rate of hydrolysis of isocyanate. Hydrolysis of 0.5 grams of diurethane in a 50 ml buffer of pH 9 at 50° C. showed complete hydrolysis in about 100 minutes.

### Example 11

### Synthesis of polyurethane from Succinic acid bis-(4-isocyanato-phenoxy carbonyl methyl) ester

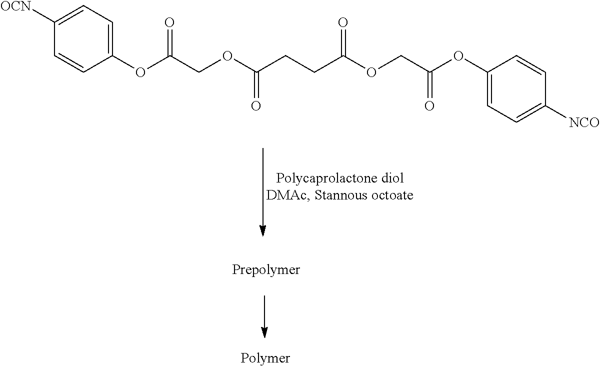

Into a clean flame dried 1000 ml 3 neck round bottom flask maintained under N₂ atmosphere at 40° C. (oil bath) was added diisocyanate (5.9 grams) dissolved in DMAc (25 ml). Into this stirring solution was added polycaprolactone diol with a MW of 2000 (10 grams) and the solution was left for stirring at RT for another 30 minutes when the temperature of the bath was raised to 40° C. 3 drops of 10% stannous octoate solution was added. Additional quantities of DMAc were added followed by stirring of the reaction mixture at 60° C. for additional 3 hours. The prepolymer formed and turned the solution viscous.

To this solution of prepolymer was added 1,4-Butanediol (675 mg) dissolved in dimethylacetamide (2 ml). To this reaction mixture was added 3 drops of 10% stannous octoate solution. The reaction mixture was left for stirring at 60° C. for 2 hour. A small fraction of the reaction mixture was poured onto a petri dish to generate film of good strength.

### Example 12

### Synthesis of polyurethane from Succinic acid bis-(4-isocyanato-phenoxy carbonyl methyl) ester

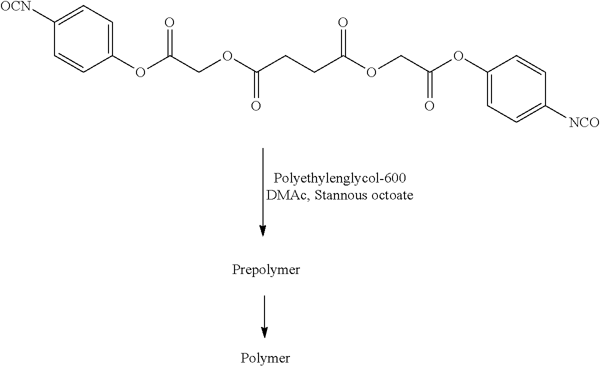

Into a clean flame dried 1000 ml 3 neck round bottom flask maintained under N₂ atmosphere at 40° C. (oil bath) was added diisocyanate (5.8 grams) dissolved in DMAc (25 ml). Into this stirring solution was added PEG with a MW of 600 (3 grams) and the solution was left for stirring at room temperature for another 30 minutes when the temperature of the bath was raised to 60° C. 3 drops of 10% stannous octoate solution was added. Additional quantities of DMAc were added followed by stirring of the reaction mixture at 60° C. for additional 3 hours. The prepolymer formed and turned the solution viscous. To this solution of prepolymer was added 1,4-butanediol (675 mg) dissolved in dimethylacetamide (2 ml). To this reaction mixture was added 3 drops of 10% stannous octoate solution. The reaction mixture was left for stirring at 60° C. for 2 hour. A small fraction of the reaction mixture was poured onto a petri dish to generate film. However the film formation did not take place.

### Example 13

### Synthesis of [2-(4-Nitro-phenoxycarbonylmethoxy)-ethoxy]acetic acid 4-nitro-phenylester

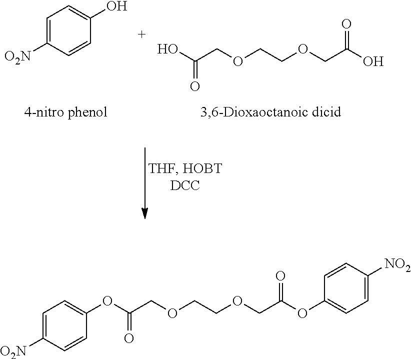

To a mixture of 4-Nitro phenol (120 grams, 862.26 mmol), 3,6-Dioxaoctanoic diacid (100 grams, 561.79 mmol), Hydroxy Benzo Triazole (38 grams, 281.06 mmol) in Tetrahydrofuran (1000 ml) under N₂ at 0° C., was added dropwise a solution of DCC (232 grams, 1.124 mmol) in THF (200 ml) and allowed to slowly come to room temperature and further stirred for 36 hours. The reaction mixture was filtered and washed with hot Tetrahydrofuran, distilled off the solvent under vacuum. The residue dissolved in Chloroform, washed with 5% Sodium bicarbonate, 1N HCl, water, dried over Sodium sulphate, distilled under reduced pressure, residue precipitated by adding Methanol, filtered, dried to yield crude[2-(4-Nitro-phenoxycarbonylmethoxy)-ethoxy]-acetic acid 4-nitro-phenylester, which was purified by recrystallising in a mixture of DMF and Methanol (1:2) to yield 108 grams of pure [2-(4-Nitro-phenoxycarbonylmethoxy)-ethoxy]-acetic acid 4-nitro-phenylester as white powder with a melting point of 123-125° C. The product was characterized by ^{I}H NMR (CDCl₃) δ 3.90 (s, 2H, CH₂), 4.50 (s, 2H, CH₂), 7.30 (d, 2H, Ar), 8.30 (d, 2H, Ar).

### Example 14

### Synthesis of 2-(4-Amino-phenoxycarbonylmethoxy)-ethoxy]acetic acid 4-amino-phenylester

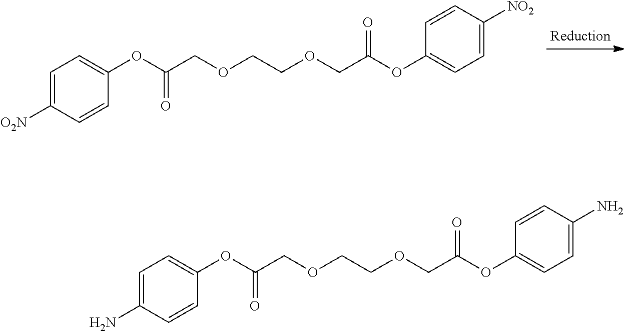 [SMILES: O=C(COCCOCC(=O)Oc1ccc([N+](=O)[O-])cc1)Oc1ccc([N+](=O)[O-])cc1 ; Nc1ccc(OC(=O)COCCOCC(=O)Oc2ccc(N)cc2)cc1]

[2-(4-Nitro-phenoxycarbonylmethoxy)-ethoxy]-acetic acid 4-nitro-phenylester (25 grams, 59.52 mmoles) was dissolved in dimethyl formamide (150 ml) in a pressure vessel, 5% Palladium carbon (5 grams, 50% wet) added and the mixture stirred under an atmosphere of Hydrogen (5 Kg) for 1 hour. The catalyst was removed by filtration, and crude product was precipitated by adding water, filtered, dried and slurried with hot diisopropyl ether to get pure [2-(4-Amino-phenoxycarbonylmethoxy)-ethoxy]-acetic acid 4-amino-phenylester (13 grams) as light brown powder with a melting point of 105-107° C. The product was characterized by ^{I}H NMR (DMSO-d₆) δ 3.75 (s, 2H, CH₂), 4.38 (s, 2H, CH₂), 5.10 (bs, 2H, NH₂), 6.60 (d, 2H, Ar), 6.80 (d, 2H, Ar). Hydrolysis of 0.5 grams of diamine in a buffer of pH 9 at 50° C. showed complete hydrolysis in 1 hour.

### Example 15

### Synthesis of [2-(4-Isocyanato-phenoxycarbonylmethoxy)-ethoxy]-acetic acid 4-isocyanato-phenyl ester

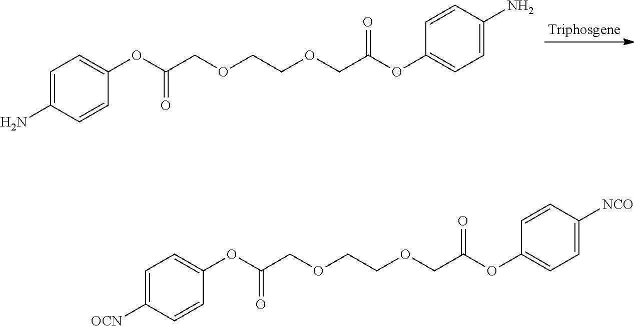

To a solution of [2-(4-Amino-phenoxycarbonylmethoxy)-ethoxy]-acetic acid 4-amino-phenylester (25 grams, 69.44 mmoles) in dry 1,4-Dioxane (500 ml) under nitrogen atmosphere was cooled to 10° C. and added a solution of Triphosgene (35 grams, 117.94 mmoles) in 1,4-Dioxane (100 ml) in one lot and heated to 85° C. temperature for 2 hours. The condenser was then arranged for distillation and solvent removed by distillation at atmospheric pressure until the volume of the reaction mixture was reduced to approximately one third. Fresh dry Dioxane (75 ml) was added and distilled off the solvents under vacuum and this process of repeated distillation was continued for two more times by adding the same quantity of 1,4-Dioxane. The residue was dissolved in Toluene (100 ml), added charcoal (5 grams), filtered hot, precipitated the [2-(4-Isocyanato-phenoxycarbonylmethoxy)-ethoxy]-acetic acid 4-isocyanato-phenyl ester by adding Hexane (200 ml), filtered redissloved in Toluene (150 ml) by heating to 100° C., filtered and precipitated by adding to cold Hexane (450 ml), filtered, dried and packed in tight container to get 14 grams of pure [2-(4-Isocyanato phenoxy carbonyl methoxy)-ethoxy]-acetic acid 4-isocyanato-phenyl ester as a white powder with a melting point of 125-128° C. The product was characterized by ^{I}H NMR (CDCl₃) δ 3.90 (s, 2H, CH₂), 4.45 (s, 2H, CH₂), 7.05-7.15 (m, 4H, Ar). The isocyanate was converted into a urethane with a melting point of 139-142.5° C. in order to determine rate of hydrolysis of isocyanate. Hydrolysis of 0.5 grams of diurethane in a buffer of pH 9 at 50° C. showed complete hydrolysis in 1 hour.

### Example 16

### Synthesis of polyurethane from [2-(4-Isocyanato-phenoxycarbonylmethoxy)-ethoxy]-acetic acid 4-isocyanato-phenyl ester

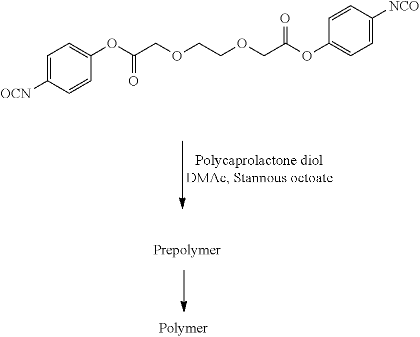

Into a clean flame dried 1000 ml 3 neck round bottom flask maintained under nitrogen atmosphere in an oil bath maintained at 50° C. was added diisocyanate (5.1 grams) dissolved in DMAc (25 ml). Into this stirring solution was added polycaprolactonediol with a molecular weight of 2000 (10 grams) and 3 drops of 10% stannous octoate solution. The solution was left for stirring at 60° C. for 4 hours. The prepolymer formed and turned the solution viscous.

Into this solution of prepolymer was added 1,4-Butanediol (675 mg) dissolved in dimethylacetamide (3 ml). To this reaction mixture was added 3 drops of 10% stannous octoate solution. The reaction mixture was left for further stirring at 60° C. for 2 hour. A small fraction of the reaction mixture was poured onto a petri dish to generate film. However the film formation did not take place.

### Example 17

### Synthesis of polyurethane from [2-(4-Isocyanato-phenoxycarbonylmethoxy)-ethoxy]-acetic acid 4-isocyanato-phenyl ester

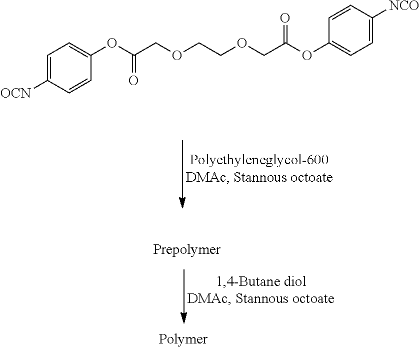

Into a clean flame dried 1000 ml 3 neck round bottom flask maintained under nitrogen atmosphere in an oil bath maintained at 50° C. was added diisocyanate (5.1 grams) dissolved in DMAc (25 ml). Into this stirring solution was added polyethyleneglycol with an average molecular weight of 600 (3 grams) and 3 drops of 10% stannous octoate solution. The solution was left for stirring at 60° C. for 4 hours. The prepolymer formed turned the solution viscous.

Into this solution of prepolymer was added 1,4-Butanediol (675 mg) dissolved in dimethylacetamide (3 ml). To this reaction mixture was added 3 drops of 10% stannous octoate solution. The reaction mixture was left for further stirring at 60° C. for 2 hour. A small fraction of the reaction mixture was poured onto a petri dish to generate film. However the film formation did not take place.

### Example 18

### Synthesis of (2,2,5-Trimethyl-[1,3]dioxan-5-yl)-carbamic acid benzyl ester

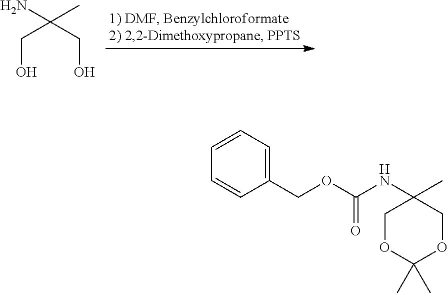

To a solution of 2-Amino-2-methyl-1,3-propanediol (100 grams, 952.38 mmol) in Dimethyl formamide (600 ml) was added Benzyl chloroformate (190 ml, 1142.85 mmol). After the mixture was stirred for 4 hours, 2,2-Dimethoxypropane (150 ml) and pyridinium-p-toluenesulfonate (10 grams) were added to this solution, which was allowed to stir for another 20 hours. Then the solution was diluted with water and extracted with ethyl acetate three times. The combined extracts were washed with water and dried over sodium sulphate, distilled and the residue precipitated with Hexane to get pure (2,2,5-Trimethyl-[1,3]dioxan-5-yl)-carbamic acid benzyl ester (97 grams) as a white powder with a melting point of 105-107° C. The product was characterized by ^{I}H NMR (CDCl₃) δ 1.28 (s, 3H, CH₃), 1.42 (s, 3H, CH₃), 1.43 (s, 3H, CH₃), 3.66 (d, 2H, CH2), 3.90 (d, 2H, CH2), 5.08 (s, 2H, CH2), 5.35 (bs, 1H, NH), 7.37 (m, 5H, Ar).

### Example 19

### Synthesis of 2,2,5-Trimethyl-[1,3]dioxan-5-yl amine

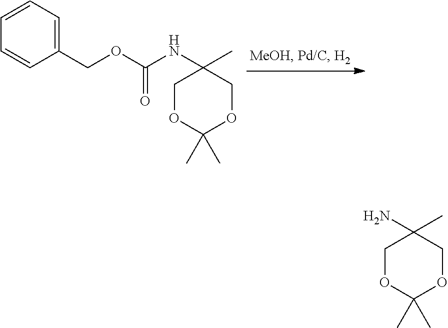

2-(6-Hydroxy-naphthalen-2-yl)-propionic acid benzyloxy carbonyl methyl ester (100 grams, 449.07 mmol) was dissolved in Methanol (500 ml) in a pressure vessel, 50% wet Palladium on carbon (10%, 15 grams) added and the mixture stirred under an atmosphere of hydrogen (4 Kg) for 2 hours. The catalyst was removed by filtration and distilled off methanol under vacuum to yield pure 2,2,5-Trimethyl-[1,3]dioxan-5-yl amine (50 grams) as yellow syrup. The product was characterized by ^{I}H NMR (CDCl₃+DMSO-d₆) δ 1.15 (s, 3H, CH₃), 1.32 (s, 3H, CH₃), 1.35 (s, 3H, CH₃), 3.75 (dd, 4H, CH₂X₂), 4.45 (bs, 2H, NH).

### Example 20

### Synthesis of Octadecanoic Acid Benzyloxy Carbonyl Methyl Ester

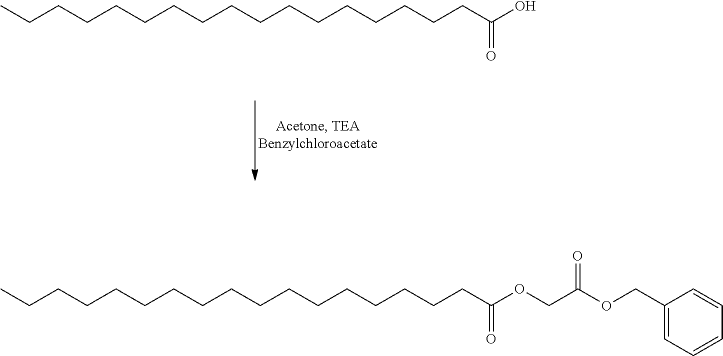 [SMILES: CCCCCCCCCCCCCCCCCC(=O)O ; CCCCCCCCCCCCCCCCCC(=O)OCC(=O)OCc1ccccc1]

To a mixture of Stearic acid (200 grams, 1.316 mol), Triethyl amine (275 ml, 1.973 mol) in Acetone (1500 ml) at room temperature was added Benzyl chloro acetate (364 grams, 1.972 mol) dropwise and stirred at reflux temperature for 5 hours. The reaction mixture poured onto cold water, extracted with Ethyl acetate, washed with 10% Sodium bicarbonate (600 ml) followed by water (600 ml), dried over Sodium sulphate, distilled under reduced pressure, residue precipitated by adding Hexane to yield pure Octadecanoic acid benzyloxy carbonyl methyl ester (180 grams) as white powder with a melting point of 48-50° C. The product was characterized by ^{I}H NMR (CDCl₃) δ 0.90 (t, 3H, CH₃), 1.30 (m, 28H, CH₂X₁₄), 1.65 (m, 2H, CH₂), 2.40 (t, 2H, CH₂), 4.70 (s, 2H, CH₂), 5.20 (s, 2H, CH₂), 7.35 (s, 5H, Ar).

### Example 21

### Synthesis of Octadecanoic Acid Carbonyl Methyl Ester

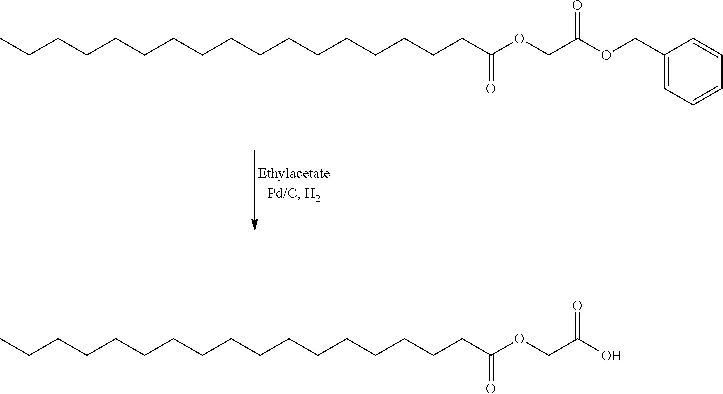 [SMILES: CCCCCCCCCCCCCCCCCC(=O)OCC(=O)OCc1ccccc1 ; CCCCCCCCCCCCCCCCCC(=O)OCC(=O)O]

Octadecanoic acid benzyloxy carbonyl methyl ester (180 grams, 416.66 mmol) was dissolved in Ethyl acetate (600 ml) in a pressure vessel, 50% wet Palladium on carbon (10%, 10 grams) added and the mixture stirred under an atmosphere of hydrogen (6 Kg) for 3 hours. The catalyst was removed by filtration and distilled off Ethyl acetate under vacuum to yield the residue, which was precipitated by adding Hexane to yield pure Octadecanoic acid carboxymethyl ester (103 grams) as white fluffy powder with a melting point of 92-93° C. The product was characterized by ^{I}H NMR (CDCl₃) δ 0.90 (t, 3H, CH₃), 1.30 (m, 28H, CH₂X14), 1.65 (m, 2H, CH₂), 2.40 (t, 2H, CH₂), 4.62 (s, 2H, CH₂).

### Example 22

### Synthesis of Octadecanoic acid (2,2,5-trimethyl-[1,3]dioxan-5-ylcarbamoyl)-methyl ester

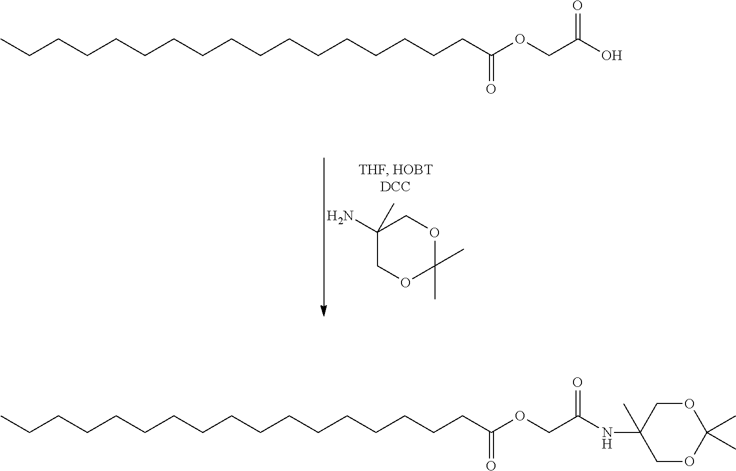

To a mixture of Octadecanoic acid carboxymethyl ester (100 grams, 292.39 mmol), 2,2,5-Trimethyl-[1,3]dioxan-5-yl amine (50.8 grams, 350.3 mmol), Hydroxy Benzotriazole (19.7 grams, 93.33 mmol) in Tetrahydrofuran (1000 ml) at 0° C., was added dropwise a solution of DCC (72.3 grams, 350.46 mmol) in Tetrahydrofuran and stirred at 0-5° C. temperature for 4 hours. The reaction mixture poured onto cold water, extracted with Ethyl acetate, washed with 5% Sodium bicarbonate (600 ml) followed by water (600 ml), dried over Sodium sulphate, distilled under reduced pressure, residue precipitated by adding Hexane to yield pure Octadecanoic acid (2,2,5-trimethyl-[1,3]dioxan-5-ylcarbamoyl)-methyl ester (108 grams) as white powder with a melting point of 70-72° C. The product was characterized by ^{I}H NMR (CDCl₃) δ 0.85 (t, 3H, CH₃), 1.25-1.40 (m, 37H, CH₂X₁₄ & CH₃X₃), 1.75 (m, 2H, CH₂), 2.35 (t, 2H, CH₂), 3.60 (d, 2H, CH₂), 3.85 (d, 2H, CH₂), 4.50 (s, 2H, CH₂), 6.50 (bs, 1H, NH).

### Example 23

### Synthesis of Octadecanoic acid (2-hydroxy-1-hydroxymethyl-1-methyl-ethylcarbamoyl)-methyl ester

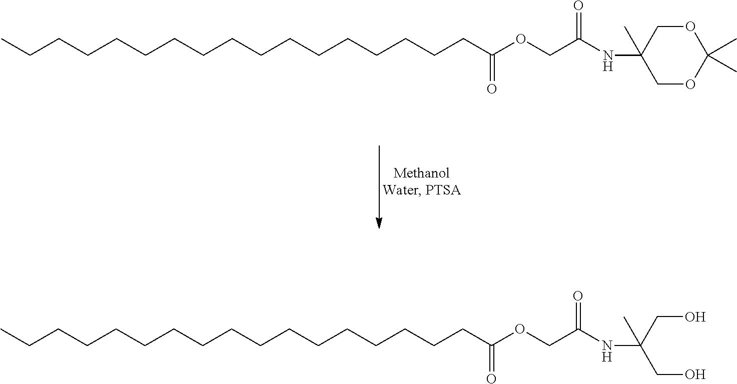

To a solution of Octadecanoic acid (2,2,5-trimethyl-[1,3]dioxan-5-ylcarbamoyl)-methyl ester (108 grams, 230.27 mmol), in a mixture of MEOH (950 ml) and water (50 ml), was added p-Toluenesulfonic acid (5 grams, 26.28 mmol) and stirred at room temperature for 1 hour. The reaction mixture poured onto cold water, extracted with Ethyl acetate, combined organic layer washed with water, dried over Sodium sulphate, distilled off the solvent under reduced pressure, to get crude compound which was precipitated by adding Hexane to yield 74 grams of pure Octadecanoic acid (2-hydroxy-1-hydroxymethyl-1-methyl-ethylcarbamoyl)-methyl ester as white powder with a MP of 87-89° C. The product was characterized by ^{I}H NMR (CDCl₃) δ 0.85 (t, 3H, CH₃), 1.25-1.35 (m, 31H, CH₂X 14 & CH₃), 1.65 (q, 2H, CH₂), 2.45 (t, 2H, CH₂), 3.65 (d, 2H, CH₂), 3.75 (d, 2H, CH₂), 4.55 (s, 2H, CH₂), 6.65 (bs, 1H, NH).

### Example 24

### Synthesis of polyester from Octadecanoic acid (2-hydroxy-1-hydroxymethyl-1-methyl-ethylcarbamoyl)-methyl ester

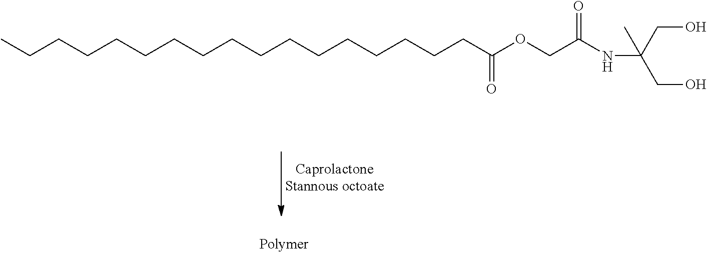 [SMILES: CCCCCCCCCCCCCCCCCC(=O)OCC(=O)NC(C)(CO)CO]

Into a clean flame dried 1000 ml 3 neck round bottom flask maintained under nitrogen atmosphere in an oil bath maintained at 50° C. was added Octadecanoic acid (2-hydroxy-1-hydroxymethyl-1-methyl-ethylc arbamoyl)-methyl ester (5.0 grams) and caprolactone (24.8 ml). Into this stirring solution was added 3 drops of 10% stannous octoate solution. The solution was left for stirring at 130° C. for 24 hours. The reaction mixture was cooled down slowly to room temperature and a high vacuum was applied. The temperature of the oil bath was increased to 50-60° C. and the solution was kept under these conditions for 7 hours to yield a light yellow thick syrup.

### Example 25

### Synthesis of polyester from Octadecanoic acid (2-hydroxy-1-hydroxymethyl-1-methyl-ethylcarbamoyl)-methyl ester

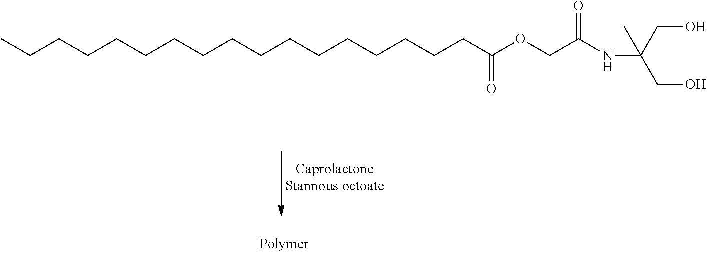 [SMILES: CCCCCCCCCCCCCCCCCC(=O)OCC(=O)NC(C)(CO)CO]

Into a clean flame dried 1000 ml 3 neck round bottom flask maintained under nitrogen atmosphere in an oil bath maintained at 50° C. was added Octadecanoic acid (2-hydroxy-1-hydroxymethyl-1-methyl-ethylcarbamoyl)-methyl ester (5.0 grams) and caprolactone (12.4 ml). Into this stirring solution was added 3 drops of 10% stannous octoate solution. The solution was left for stirring at 130° C. for 24 hours. The reaction mixture was cooled down slowly to room temperature and a high vacuum was applied. The temperature of the oil bath was increased to 50-60° C. and the solution was kept under these conditions for 7 hours to yield a light yellow thick syrup.

### Example 26

### Synthesis of Octadecanoic acid (2,2,5-trimethyl-[1,3]dioxan-5-yl)-amide

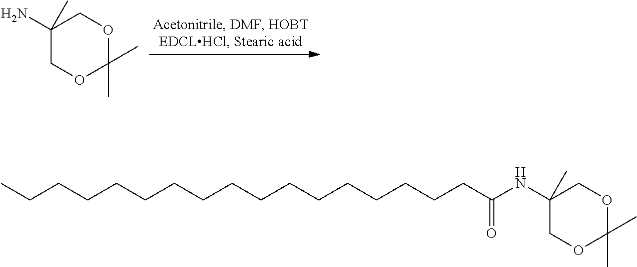

To a mixture of Stearic acid (28.5 grams), 2,2,5-Trimethyl-[1,3]dioxan-5-yl amine (30 grams), Hydroxy Benzo Triazole (12.6 grams) in Acetonitrile (300 ml) and Dimethyl formamide (100 ml) at 0° C., was added EDCl.HCl (43 grams) in small lots and stirred at 0-5° C. temperature for 1 hour, later heated to 50° C. for two hours. The reaction mixture poured onto cold water:methanol (9:1; 500 ml), stirred for 30 minutes, filtered separated solid and dried to get 42 grams of pure Octadecanoic acid (2,2,5-trimethyl-[1,3]dioxan-5-yl)-amide as a white powder with a melting point of 74-76° C. The product was characterized by ^{I}H NMR (CDCl₃) δ 0.86 (t, 3H, CH₃), 1.26 (m, 31H, CH₂X₁₄ & CH₃), 1.40 (s, 3H, CH₃), 1.41 (s, 3H, CH₃), 1.60 (m, 2H, CH₂), 2.18 (t, 2H, COCH₂), 3.60 (d, 2H, CH₂), 3.92 (d, 2H, CH₂), 5.78 (bs, 1H, NH).

### Example 27

### Synthesis of Octadecanoic acid (2-hydroxy-1-hydroxymethyl-1-methyl-ethyl)-amide

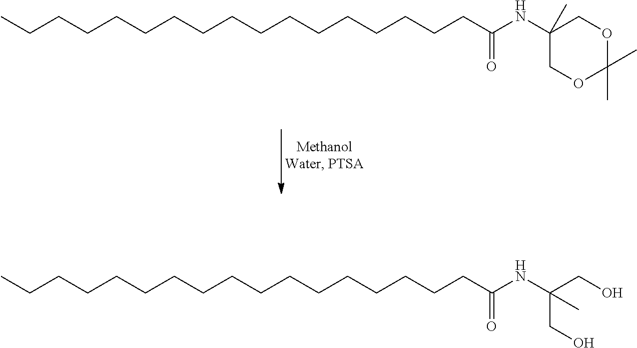

To a solution of Octadecanoic acid (2,2,5-trimethyl-[1,3]dioxan-5-yl)-amide (42 grams, 150.53 mmol), in a mixture of Methanol (420 ml) and water (20 ml), was added p-Toluenesulfonic acid (1 grams, 5.25 mmol) and stirred at room temperature for 4 hours. The reaction mixture poured onto cold water, extracted with Ethyl acetate, combined organic layer washed with water, dried over Sodium sulphate, distilled off 60% of the solvent under reduced pressure, pour onto cold water, extracted with Ethyl acetate, wash the organic layer with water, dried over Sodium sulphate and distilled under vacuum to get crude compound which was purified by recrystallisation using Ethyl acetate and precipitating with Hexane to get 18 grams of Octadecanoic acid (2-hydroxy-1-hydroxymethyl-1-methyl-ethyl)-amide as white powder with a melting point of 74-76° C. The product was characterized by ^{I}H NMR (CDCl₃) δ 0.86 (t, 3H, CH₃), 1.24 (m, 31H, CH₂X₁₄ & CH₃), 1.56 (m, 2H, CH₂), 2.17 (t, 2H, COCH₂), 3.57 (d, 2H, CH₂), 3.70 (d, 2H, CH₂), 4.17 (bs, 2H, OH), 5.96 (bs, 1H, NH).

### Example 28

### Synthesis of polymer from Octadecanoic acid (2-hydroxy-1-hydroxymethyl-1-methyl-ethyl)-amide

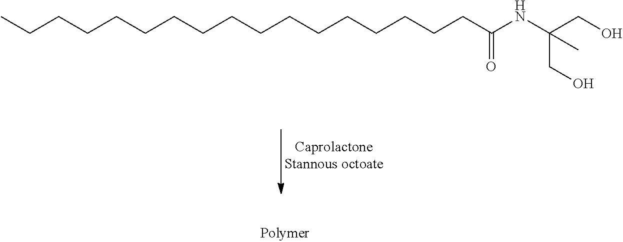 [SMILES: CCCCCCCCCCCCCCCCCC(=O)NC(C)(CO)CO]

Into a clean flame dried 1000 ml 3 neck round bottom flask maintained under nitrogen atmosphere in an oil bath maintained at room temperature was added Octadecanoic acid (2-hydroxy-1-hydroxymethyl-1-methyl-ethyl)-amide (5.0 grams) and caprolactone (28.5 ml). Into this stirring solution was added 3 drops of 10% stannous octoate solution. The solution was left for stirring at 140° C. for 12 hours. The reaction mixture was cooled down slowly to room temperature and a high vacuum was applied. The temperature of the oil bath was increased to 50° C. and the solution was kept under these conditions for 3 hours to yield 30 grams of light yellow thick syrup.

### Example 29

### Synthesis of 5-Ethyl-2,2-dimethyl-[1,3]dioxane-5-carboxylic acid

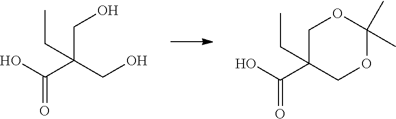

A mixture of 2,2-bis(hydroxymethyl)butyric acid (200 grams, 1.350 mol), 2,2-Dimethoxy propane (215 ml, 1.750 mol) and PPTS (20 grams) in Toluene (1000 ml) was stirred at 80° C. temperature with distillation set up to collect the methanol formed for 5 hours. The reaction mixture cooled to room temperature, washed with water (600 ml), dried over Sodium sulphate, distilled under reduced pressure, and the residue precipitated with Hexane, filtered dried to give pure 5-Ethyl-2,2-dimethyl-[1,3]dioxane-5-carboxylic acid (140 grams) as a white powder with a melting point of 79-81° C. The product was characterized by ^{I}H NMR (CDCl₃) δ 0.90 (t, 3H, CH₃), 1.40 (s, 6H, CH₃X₂), 1.70 (q, 2H, CH₂), 3.70 (d, 2H, CH₂), 4.15 (d, 2H, CH₂).

### Example 30

### Synthesis of 5-Ethyl-2,2-dimethyl-[1,3]dioxane-5-carboxylic acid benzyloxy carbonyl methyl ester

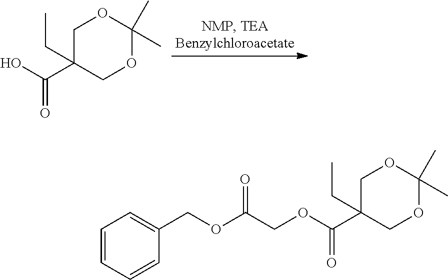 [SMILES: CCC1(C(=O)O)COC(C)(C)OC1 ; CCC1(C(=O)OCC(=O)OCc2ccccc2)COC(C)(C)OC1]

To a mixture of 5-Ethyl-2,2-dimethyl-[1,3]dioxane-5-carboxylic acid (50 grams, 265.95 mmol), Triethyl amine (32.2 grams, 319.14 mmol) in N-Methyl-2-pyrrolidone (250 ml) at 0-5° C. was added benzyl chloro acetate (58.6 grams, 317.44 mmol) dropwise and stirred at room temperature for over night. The reaction mixture poured onto cold water, extracted with Ethyl acetate, washed with water (600 ml), dried over Sodium sulphate, distilled under reduced pressure, purified by column chromatography using Hexane:Ethyl acetate (8:2) to yield pure 5-Ethyl-2,2-dimethyl-[1,3]dioxane-5-carboxylic acid benzyloxy carbonyl methyl ester (53 grams) as light yellow syrup. The product was characterized by ^{I}H NMR (CDCl₃) δ 0.80 (t, 3H, CH₃), 1.28 (s, 3H, CH₃), 1.30 (s, 3H, CH₃), 1.65 (q, 2H, CH₂), 3.60 (d, 2H, CH₂), 4.05 (d, 2H, CH₂), 4.60 (s, 2H, CH₂), 5.10 (s, 2H, CH₂), 7.25 (s, 5H, Ar).

### Example 31

### Synthesis of 5-Ethyl-2,2-dimethyl-[1,3]dioxane-5-carboxylic acid carboxy methyl ester

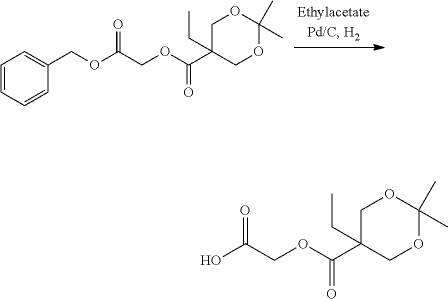 [SMILES: CCC1(C(=O)OCC(=O)OCc2ccccc2)COC(C)(C)OC1 ; CCC1(C(=O)OCC(=O)O)COC(C)(C)OC1]

5-Ethyl-2,2-dimethyl-[1,3]dioxane-5-carboxylic acid benzyloxy carbonyl methyl ester (53 grams, 157.73 mmol) was dissolved in Ethyl acetate (350 ml) in a pressure vessel, 50% wet Palladium on carbon (10%, 20 grams) added and the mixture stirred under an atmosphere of hydrogen (5 Kg) for overnight. The catalyst was removed by filtration and distilled off Ethyl acetate under vacuum to yield pure 5-Ethyl-2,2-dimethyl-[1,3]dioxane-5-carboxylic acid carboxymethyl ester (40 grams) as light yellow syrup. The product was characterized by ^{I}H NMR (CDCl₃) δ 0.85 (t, 3H, CH₃), 1.28 (s, 3H, CH₃), 1.30 (s, 3H, CH₃), 1.65 (q, 2H, CH₂), 3.60 (d, 2H, CH₂), 4.10 (d, 2H, CH₂), 4.60 (s, 2H, CH₂)

### Example 32

### Synthesis of 5-Ethyl-2,2-dimethyl-[1,3]dioxane-5-carboxylic acid octadecyloxy carbonyl methyl ester

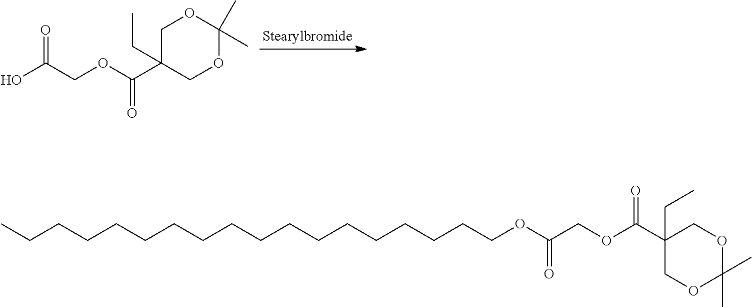 [SMILES: CCC1(C(=O)OCC(=O)O)COC(C)(C)OC1 ; CCCCCCCCCCCCCCCCCCOC(=O)COC(=O)C1(CC)COC(C)(C)OC1]

To a mixture of 5-Ethyl-2,2-dimethyl-[1,3]dioxane-5-carboxylic acid carboxymethyl ester (53 grams, 215.44 mmol), Triethyl amine (37.6 ml, 269.76 mmol) in Acetone (600 ml) was added Stearyl bromide (60 grams, 179.96 mmol) and stirred at reflux room temperature for 48 hours. The reaction mixture poured onto cold water, extracted with Ethyl acetate, washed with water (600 ml), dried over Sodium sulphate, distilled under reduced pressure, purified by column chromatography using Hexane:Ethyl acetate (8:2) to yield pure 5-Ethyl-2,2-dimethyl-[1,3]dioxane-5-carboxylic acid octadecyloxy carbonyl methyl ester (42 grams) as light yellow syrup. The product was characterized by ^{I}H NMR (CDCl₃) δ 0.90 (m, 6H, CH₃X₂), 1.25 (s, 6H, CH₃X₂), 1.45 (m, 32H, CH₂X₁₆), 1.70 (q, 2H, CH₂), 1.85 (t, 2H, CH₂), 3.80 (d, 2H, CH₂), 4.20 (m, 4H, CH₂X₂), 4.70 (s, 2H, CH₂).

### Example 33

### Synthesis of 2,2-Bis-hydroxymethyl-butyric acid octadecyloxy carbonyl methyl ester

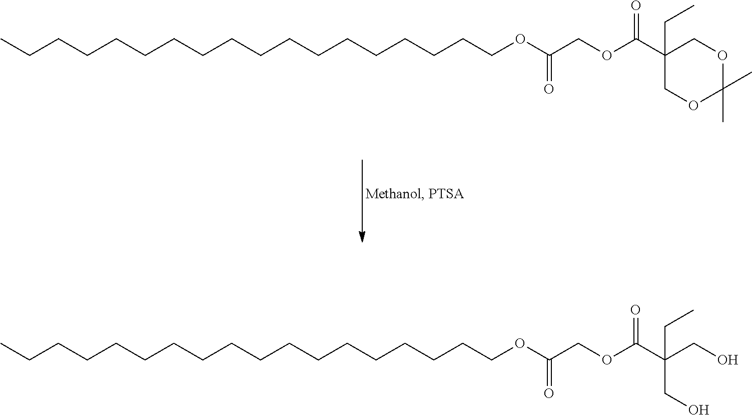

To a solution of 5-Ethyl-2,2-dimethyl-[1,3]dioxane-5-carboxylic acid octadecyloxy carbonyl methyl ester (42 grams, 84.33 mmol), in a mixture of methanol (420 ml) and water (20 ml), was added p-Toluenesulfonic acid (4.2 grams, 22.08 mmol) and stirred at room temperature for 48 hours. The reaction mixture poured onto cold water, extracted with ethyl acetate, combined organic layer washed with water, dried over Sodium sulphate, distilled off the solvent under reduced pressure, to get crude compound which was purified by column chromatography using Hexane:Ethyl acetate to yield 10 grams of pure 2,2-Bis-hydroxymethyl-butyric acid octadecyloxy carbonyl methyl ester as white powder with a melting point of 57-59.5° C. The product was characterized by ^{I}H NMR (CDCl₃) δ 0.95 (m, 6H, CH₃X₂), 1.30 (m, 32H, CH₂X₁₆), 1.65 (m, 4H, CH₂X₂), 3.75 (d, 2H, CH₂), 3.95 (d, 2H, CH₂), 4.15 (t, 2H, CH₂), 4.70 (s, 2H, CH₂).

### Example 34

### Synthesis of polymer from 2,2-Bis-hydroxymethyl-butyric acid octadecyloxy carbonyl methyl ester

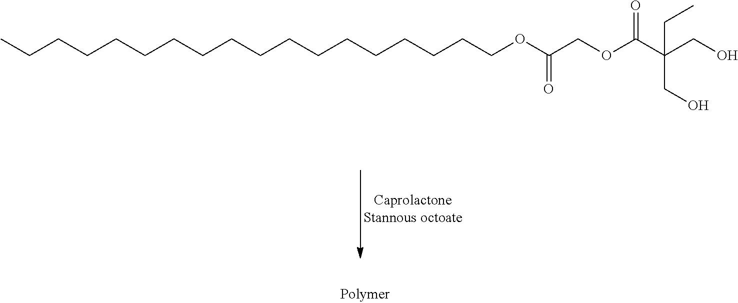 [SMILES: CCCCCCCCCCCCCCCCCCOC(=O)COC(=O)C(CC)(CO)CO]

Into a clean flame dried 1000 ml 3 neck round bottom flask maintained under nitrogen atmosphere in an oil bath maintained at room temperature was added 2,2-Bis-hydroxymethyl-butyric acid octadecyloxy carbonyl methyl ester (5.0 grams) and caprolactone (11.5 ml). Into this stirring solution were added 10 drops of 10% stannous octoate solution. The solution was left for stirring at 130° C. for 20 hours. The reaction mixture was cooled down slowly to room temperature and a high vacuum was applied. The temperature of the oil bath was increased to 50 to 60° C. and the solution was kept under these conditions for 7 hours to yield 16 grams of light yellow thick syrup.

### Example 35

### Synthesis of polymer from 2,2-Bis-hydroxymethyl-butyric acid octadecyloxy carbonyl methyl ester

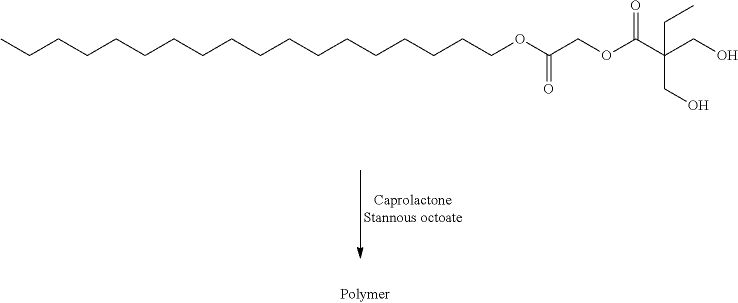 [SMILES: CCCCCCCCCCCCCCCCCCOC(=O)COC(=O)C(CC)(CO)CO]

Into a clean flame dried 1000 ml 3 neck round bottom flask maintained under nitrogen atmosphere in an oil bath maintained at room temperature was added 2,2-Bis-hydroxymethyl-butyric acid octadecyloxy carbonyl methyl ester (5.0 grams) and caprolactone (23 ml). Into this stirring solution were added 10 drops of 10% stannous octoate solution. The solution was left for stirring at 130° C. for 20 hours. The reaction mixture was cooled down slowly to room temperature and a high vacuum was applied. The temperature of the oil bath was increased to 50 to 60° C. and the solution was kept under these conditions for 7 hours to yield 27 grams of light yellow thick syrup.

### Example 36

### Synthesis of (2,2-Bis-hydroxymethyl-butyric acid octadecyl ester)

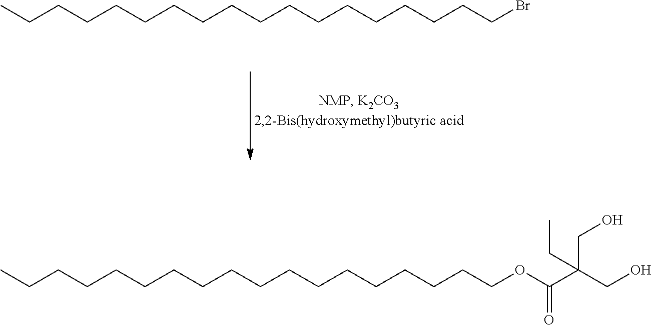 [SMILES: CCCCCCCCCCCCCCCCCCBr ; CCCCCCCCCCCCCCCCCCOC(=O)C(CC)(CO)CO]

To a mixture of 2,2-Bis(hydroxymethyl)butyric acid (49 grams), Potassium carbonate (52 grams) in N-Methyl-2-pyrrolidone (200 ml) was added Stearyl bromide (50 grams) in small lots and stirred at room temperature under Nitrogen atmosphere for 48 hours. The reaction mixture poured onto cold water, extracted with Diisopropyl ether, washed with water (300 ml), dried over Sodium sulphate, distilled under reduced pressure, purified by column chromatography using Hexane:Ethyl acetate (8:2) followed by precipitating in Hexane to give pure 2,2-Bis-hydroxymethyl-butyric acid octadecyl ester (18 grams) as a white powder with a melting point of 46-48° C. The product was characterized by ^{I}H NMR (CDCl₃) δ 0.82 (m, 6H, CH₃X₂), 1.25 (m, 32H, CH₂X₁₆), 1.50 (q, 2H, CH₂), 2.75 (bs, 2H, OH), 3.72 (d, 2H, CH₂), 4.05 (d, 2H, CH₂), 4.20 (t, 2H, CH₂).

### Example 37

### Synthesis of polymer from (2,2-Bis-hydroxymethyl-butyric acid octadecyl ester)

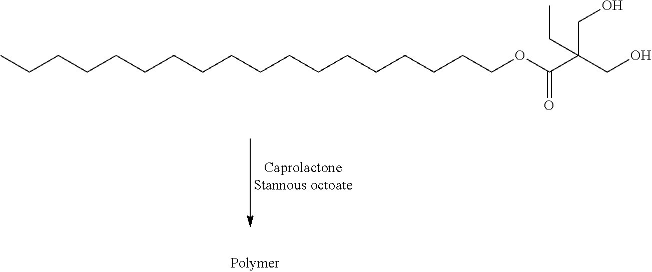 [SMILES: CCCCCCCCCCCCCCCCCCOC(=O)C(CC)(CO)CO]

Into a clean flame dried 1000 ml 3 neck round bottom flask maintained under nitrogen atmosphere in an oil bath maintained at room temperature was added 2,2-Bis-hydroxymethyl-butyric acid octadecyl ester (5.0 grams) and caprolactone (26.4 ml). Into this stirring solution were added 13 drops of 10% stannous octoate solution. The solution was left for stirring at 130° C. for 26 hours. The reaction mixture was cooled down slowly to room temperature and a high vacuum was applied. The temperature of the oil bath was increased to 50 to 60° C. and the solution was kept under these conditions for 7 hours to yield 30 grams of light yellow thick syrup which turned into an off white gel after being cooled down to room temperature.

### Example 38

### Synthesis of polymer from (2,2-Bis-hydroxymethyl-butyric acid octadecyl ester)

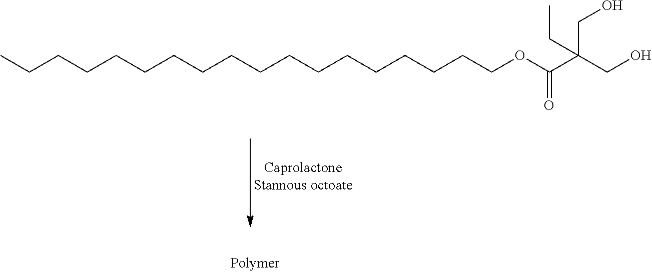 [SMILES: CCCCCCCCCCCCCCCCCCOC(=O)C(CC)(CO)CO]

Into a clean flame dried 1000 ml 3 neck round bottom flask maintained under nitrogen atmosphere in an oil bath maintained at room temperature was added 2,2-Bis-hydroxymethyl-butyric acid octadecyl ester (5.0 grams) and caprolactone (13.2 ml). Into this stirring solution were added 13 drops of 10% stannous octoate solution. The solution was left for stirring at 130° C. for 26 hours. The reaction mixture was cooled down slowly to room temperature and a high vacuum was applied. The temperature of the oil bath was increased to 50 to 60° C. and the solution was kept under these conditions for 7 hours to yield 16 grams of light yellow thick syrup which turned into an off white low melting semi-solid after being cooled down to room temperature.

### Example 39

### Synthesis of 2-Acetoxy-benzoic acid benzyloxy carbonyl methyl ester

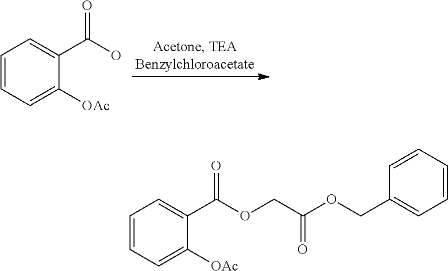 [SMILES: CC(=O)Oc1ccccc1C(=O)O ; CC(=O)Oc1ccccc1C(=O)OCC(=O)OCc1ccccc1]

To a mixture of Aspirin (25 grams, 138.77 mmol), Triethylamine (29 ml, 208.06 mmol) in acetone (250 ml) was added Benzyl chloro acetate (30.75 grams, 166.56 mmol) drop wise and stirred at 50° C. temperature for five hours. The reaction mixture poured onto cold water, crude product filtered, dried and purified by recrystallising from a mixture of Chloroform:Hexane (1:4) to yield pure 2-Acetoxy-benzoic acid benzyloxy carbonyl methyl ester (25 grams) as a white powder with a melting point of 91-92.5° C. The product was characterized by ^{I}H NMR (CDCl₃) δ 2.30 (s, 3H, OAc), 4.82 (s, 2H, CH₂), 5.20 (s, 2H, CH₂), 7.10 (d, 1H, Ar), 7.32 (m, 6H, Ar), 7.56 (t, 1H, Ar), 8.18 (d, 1H, Ar).

### Example 40

### Synthesis of 2-Acetoxy-benzoic acid carboxymethyl ester

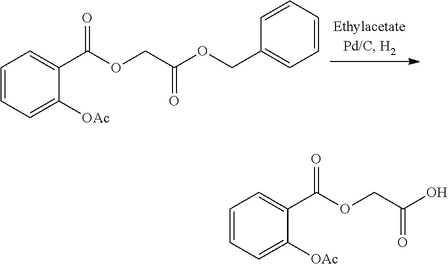 [SMILES: CC(=O)Oc1ccccc1C(=O)OCC(=O)OCc1ccccc1 ; CC(=O)Oc1ccccc1C(=O)OCC(=O)O]

2-Acetoxy-benzoic acid benzyloxy carbonyl methyl ester (50 grams, 152.43 mmol) was dissolved in Ethyl acetate (150 ml) in a pressure vessel, 50% wet Palladium on carbon (10%, 10 grams) added and the mixture stirred under an atmosphere of hydrogen (4 Kg) for 14 hours. The catalyst was removed by filtration and distilled off the Ethyl acetate under vacuum and precipitated the crude by adding Hexane, filtered dried and purified by recrystallising in a mixture of Ethyl acetate:Hexane (1:6) to yield pure 2-Acetoxy-benzoic acid carboxymethyl ester (32 grams) as a white powder with a melting point of 130-131.5° C. The product was characterized by ^{I}H NMR (DMSO-d₆) δ 2.28 (s, 3H, OAc), 4.8 (s, 2H, CH₂), 7.24 (d, 1H, Ar), 7.55 (t, 1H, Ar), 7.74 (t, 1H, Ar), 8.10 (d, 1H, Ar), 13.20 (bs, 1H, COOH).

### Example 41

### Synthesis of 2-Acetoxy-benzoic acid 4-bromo-butoxy carbonyl methyl ester

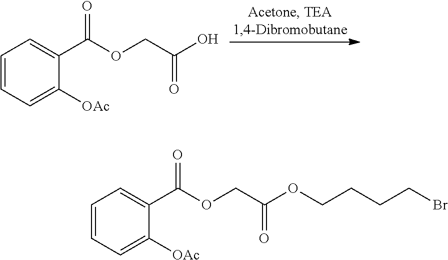 [SMILES: CC(=O)Oc1ccccc1C(=O)OCC(=O)O ; CC(=O)Oc1ccccc1C(=O)OCC(=O)OCCCCBr]

To a mixture of 2-Acetoxy-benzoic acid carboxymethyl ester (30 grams, 126.05 mmol), Triethylamine (26.5 ml, 190.12 mmol) in acetone (200 ml) was added 1,4-Dibromo butane (109 grams, 504.86 mmol) drop wise and stirred at room temperature for 24 hours. The reaction mixture poured onto cold water, crude product was extracted into Dichloromethane, dried over Sodium sulphate, distilled off the solvent under reduced pressure and the residue was purified by column chromatography using Hexane as eluant to yield 32 grams of 2-Acetoxy-benzoic acid 4-bromo-butoxy carbonyl methyl ester as a light yellow liquid. The product was characterized by ^{I}H NMR (CDCl₃) δ 1.88 (m, 4H, CH₂X₂), 2.32 (s, 3H, OAc), 3.39 (t, 2H, CH₂), 4.20 (t, 2H, CH₂), 4.75 (s, 2H, CH₂), 7.10 (d, 1H, Ar), 7.23 (t, 1H, Ar), 1H, Ar), 8.10 (d, 1H, Ar).

### Example 42

### Synthesis of 2-Methyl-benzoic acid 4-(2,2-bis-hydroxymethyl-butyryloxy)-butoxy carbonyl methyl ester

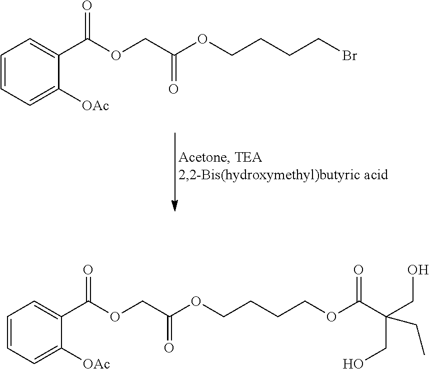 [SMILES: CC(=O)Oc1ccccc1C(=O)OCC(=O)OCCCCBr ; CCC(CO)(CO)C(=O)OCCCCOC(=O)COC(=O)c1ccccc1OC(C)=O]

To a mixture of 2-Acetoxy-benzoic acid 4-bromo-butoxy carbonyl methyl ester (40.21 mmol), Triethylamine (121.97 mmol) in acetone (150 ml) under nitrogen atmosphere was added 2,2-Bis(hydroxy methyl) butyric acid (80.99 mmol) drop wise and stirred at room temperature for 24 hours. The reaction mixture was poured onto cold water, crude product extracted into dichloro methane and washed with 5% Sodium bicarbonate (75 ml), water (90 ml) dried over sodium sulphate, distilled off the solvent under reduced pressure and the residue was purified by column chromatography using hexane:ethyl acetate (8:2) as an eluant to obtain 2-Methyl-benzoic acid 4-(2,2-bis-hydroxymethyl-butyryloxy)-butoxy carbonyl methyl ester as a light yellow liquid. The product was characterized by ^{I}H NMR (CDCl₃) δ 1.88 (m, 4H, CH₂X 2), 2.32 (s, 3H, OAc), 3.39 (t, 2H, CH₂), 4.20 (t, 2H, CH₂), 4.75 (s, 2H, CH₂), 7.10 (d, 1H, Ar), 7.23 (t, 1H, Ar), 7.50 (t, 1H, Ar), 8.10 (d, 1H, Ar).

### Example 43

### Synthesis of 2-(6-Methoxy-naphthalen-2-yl)-propionic acid benzyloxy carbonyl methyl ester

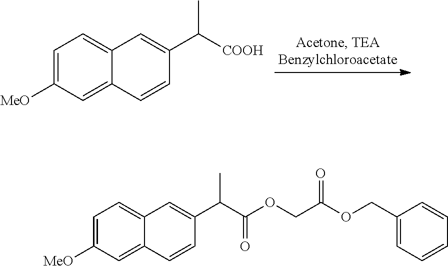 [SMILES: COc1ccc2cc(C(C)C(=O)O)ccc2c1 ; COc1ccc2cc(C(C)C(=O)OCC(=O)OCc3ccccc3)ccc2c1]

To a mixture of Naproxen (25 grams, 108.56 mmol), Triethylamine (23 ml, 165.01 mmol) in acetone (150 ml) was added Benzyl chloro acetate (24 grams, 132.15 mmol) drop wise and stirred at 50° C. temperature for three hours. The reaction mixture poured onto cold water, crude filtered, dried and purified by recrystallising from a mixture of Ethyl acetate:Hexane (1:5) to yield pure 2-(6-Methoxy-naphthalen-2-yl)-propionic acid benzyloxy carbonyl methyl ester (39 grams) as a white powder with a melting point of 95.3-97.3° C. The product was characterized by ^{I}H NMR (CDCl₃) δ 1.60 (d, 3H, CH₃), 3.80 (m, 4H, CH and OCH₃), 4.56 (q, 2H, OCH2), 5.12 (q, 2H, OCH2), 7.06 (m, 2H, Ar), 7.30 (m, 6H, Ar), 7.64 (m, 3H, Ar).

### Example 44

### Synthesis of 2-(6-Methoxy-naphthalen-2-yl)-propionic acid carboxymethyl ester

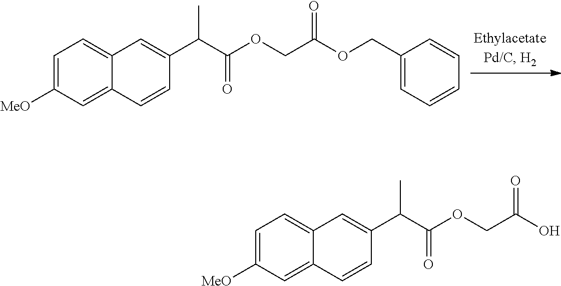 [SMILES: COc1ccc2cc(C(C)C(=O)OCC(=O)OCc3ccccc3)ccc2c1 ; COc1ccc2cc(C(C)C(=O)OCC(=O)O)ccc2c1]

2-(6-Methoxy-naphthalen-2-yl)-propionic acid benzyloxy carbonyl methyl ester (45 grams, 119.04 mmol) was dissolved in Ethyl acetate (200 ml) in a pressure vessel, 50% wet Palladium on carbon (10%, 9 grams) added and the mixture stirred under an atmosphere of hydrogen (4 Kg) for overnight. The catalyst was removed by filtration and distilled off the Ethyl acetate under vacuum and precipitated the crude by adding Hexane, filtered, dried and purified by recrystallising in a mixture of Ethyl acetate:Hexane (1:5) to yield pure 246-Methoxy-naphthalen-2-yl)-propionic acid carboxymethyl ester (30 grams) as a white powder with a melting point of 131-132.5° C. The product was characterized by ^{I}H NMR (CDCl₃) δ 1.60 (d, 3H, CH₃), 3.88 (s, 1H, OCH₃), 3.94 (m, 1H, CH), 4.54 (q, 2H, OCH2), 7.06 (m, 2H, Ar), 7.39 (d, 1H, Ar), 7.64 (m, 3H, Ar).

### Example 45

### Synthesis of 2-(6-Methoxy-naphthalen-2-yl)-propionic acid 4-bromo-butoxy carbonyl methyl ester

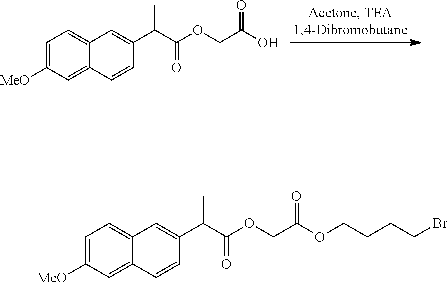 [SMILES: COc1ccc2cc(C(C)C(=O)OCC(=O)O)ccc2c1 ; COc1ccc2cc(C(C)C(=O)OCC(=O)OCCCCBr)ccc2c1]

To a mixture of 2-(6-Methoxy-naphthalen-2-yl)-propionic acid carboxymethyl ester (30 grams, 104.16 mmol), Triethylamine (21.9 ml, 157.12 mmol) in acetone (200 ml) was added 1,4-Dibromo butane (90 grams, 416.85 mmol) drop wise and stirred at room temperature for 24 hours. The reaction mixture was poured onto cold water, crude product was extracted into dichloromethane, dried over Sodium sulphate, distilled off the solvent under reduced pressure and the residue was purified by column chromatography using Hexane as eluant to yield 18 grams of 2-(6-Methoxy-naphthalen-2-yl)-propionic acid 4-bromo-butoxy carbonyl methyl ester as a light brown syrup. The product was characterized by ^{I}H NMR (CDCl₃) δ 1.52 (d, 3H, CH₃), 1.62 (m, 4H, CH₂X₂), 3.16 (t, 2H, CH₂), 3.82 (m, 4H, CH and OCH₃), 3.98 (t, 2H, CH₂), 4.46 (q, 2H, OCH2), 7.00 (m, 2H, Ar), 7.30 (d, 1H, Ar), 7.58 (m, 3H, Ar).

### Example 46

### Synthesis of 2,2-Bis-hydroxymethyl-butyric acid 4-{2-[2-(6-methoxy-naphthalen-2-yl)-propionyloxy]-acetoxy}-butyl ester

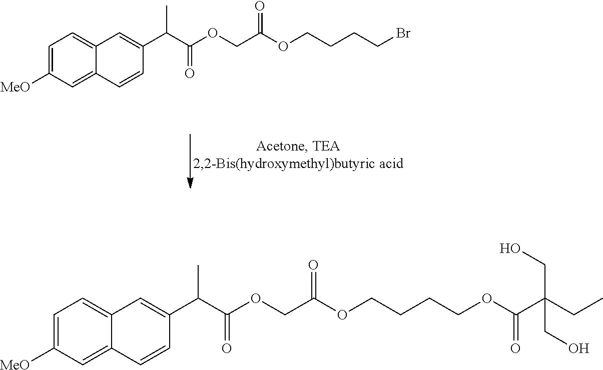 [SMILES: COc1ccc2cc(C(C)C(=O)OCC(=O)OCCCCBr)ccc2c1 ; CCC(CO)(CO)C(=O)OCCCCOC(=O)COC(=O)C(C)c1ccc2cc(OC)ccc2c1]

To a mixture of 2-(6-Methoxy-naphthalen-2-yl)-propionic acid 4-bromo-butoxy carbonyl methyl ester (15 grams, 35.46 mmol), Triethylamine (12.5 ml, 89.68 mmol) in acetone (200 ml) was added 2,2-Bis(hydroxy methyl) butyric acid (10.5 grams, 70.87 mmol) drop wise and stirred at reflux temperature for 48 hours. The reaction mixture poured onto cold water and crude product extracted into dichloro methane, washed with 5% Sodium bicarbonate solution (100 ml), water (100 ml), dried over sodium sulphate, distilled off the solvent under reduced pressure and the residue was double purified by column chromatography using Hexane:Ethyl acetate as eluant to yield 6 grams of 2,2-Bis-hydroxymethyl-butyric acid 4-{2-[2-(6-methoxy-naphthalen-2-yl)-propionyloxy]-acetoxy}-butyl ester as a light yellow syrup. The product was characterized by ^{I}H NMR (CDCl₃+DMSO-d₆) δ 0.81 (t, 3H, CH₃), 1.50 (q, 2H, CH₂), 1.62 (m, 7H, CH₃ & CH₂X₂), 3.62 (d, 2H, CH₂), 3.85 (s, 3H, OCH₃), 3.95 (m, 3H, CH₂ & CH), 4.62 (q, 2H, OCH2), 7.15 (m, 2H, Ar), 7.45 (dd, 1H, Ar), 7.65 (m, 3H, Ar).

### Example 47

### Synthesis of Succinic Acid Mono Benzyl Ester

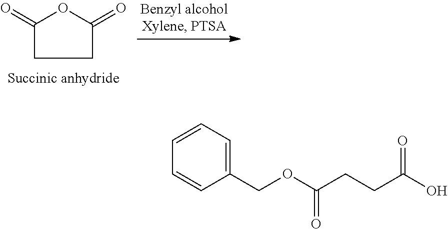 [SMILES: O=C1CCC(=O)O1 ; O=C(O)CCC(=O)OCc1ccccc1]

A solution of Succinic anhydride (184 grams, 1.84 mol), Benzyl alcohol (200 grams, 1.849 mol) and PTSA (1 gram, 5.25 mmol) in Xylene (1200 ml) was stirred at reflux temperature for 5 hours. The reaction mixture was cooled to room temperature, poured onto 10% Sodium bicarbonate solution (2500 ml), aqueous layer washed with Ethyl acetate (500 ml), pH made acidic with dil HCl, extracted with Chloroform, dried over Sodium sulphate, distilled under reduced pressure, precipitated the residue by using Hexane to give pure Succinic acid mono benzyl ester (250 grams) as white powder with a melting point of 61-63° C. The product was characterized by ^{I}H NMR (CDCl₃) δ 2.70 (m, 4H, CH₂X₂), 5.20 (s, 2H, CH₂), 7.35 (m, 5H, Ar), 10.25 (bs, 1H, COOH).

### Example 48

### Synthesis of Succinic Acid Benzyl Ester Benzyloxy Carbonyl Methyl Ester

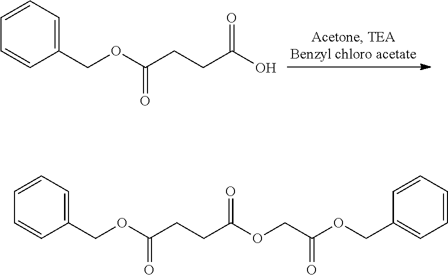 [SMILES: O=C(O)CCC(=O)OCc1ccccc1 ; O=C(CCC(=O)OCc1ccccc1)OCC(=O)OCc1ccccc1]

To a mixture of Succinic acid mono benzyl ester (250 grams, 1.201 mol), Triethyl amine (250 ml, 1.793 mol) in Acetone (2500 ml) at room temperature was added Benzyl chloro acetate (222 grams, 1.202 mol) drop wise and stirred at room temperature for 40 hours. The reaction mixture filtered to remove the salts, solvent distilled off, residue dissolved in Chloroform (1000 ml), washed with 5% Sodium bicarbonate, water, dried over sodium sulphate, distilled off the solvent under reduced pressure and precipitated the pure compound by adding Hexane to give pure Succinic acid benzyl ester benzyloxy carbonyl methyl ester (360 grams) as white powder with a melting point of 53-54° C. The product was characterized by ^{I}H NMR (CDCl₃) δ 2.80 (m, 4H, CH₂X₂), 4.75 (s, 2H, CH₂), 5.10 (s, 2H, CH₂), 5.20 (s, 2H, CH₂), 7.30 (s, 5H, Ar).

### Example 49

### Synthesis of Succinic Acid Mono Carboxy Methyl Ester

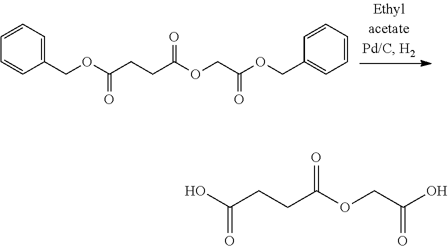 [SMILES: O=C(CCC(=O)OCc1ccccc1)OCC(=O)OCc1ccccc1 ; O=C(O)CCC(=O)OCC(=O)O]

Succinic acid benzyl ester benzyloxy carbonyl methyl ester (360 grams, 1.011 mol) was dissolved in Ethyl acetate (1000 ml) in a pressure vessel, 50% wet Palladium on carbon (5%, 50 grams) added and the mixture stirred under an atmosphere of hydrogen (8 Kg) for 5 hours. The catalyst was removed by filtration and distilled off Ethyl acetate under vacuum, the residue precipitated with Hexane, filtered, slurried in Diisopropyl ether to get pure Succinic acid mono carboxy methyl ester (146 grams) as white powder with a melting point of 115-116° C. The product was characterized by ^{I}H NMR (DMSO-d₆) δ 2.55 (m, 4H, CH₂X₂), 4.60 (s, 2H, CH₂).

### Example 50

### Synthesis of Succinic Acid Mono Carboxy Methyl Ester

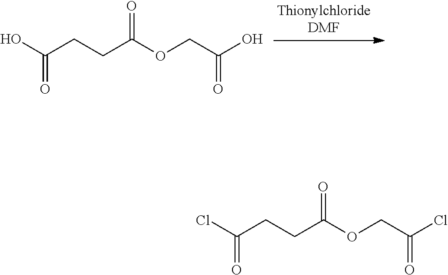 [SMILES: O=C(O)CCC(=O)OCC(=O)O ; O=C(Cl)CCC(=O)OCC(=O)Cl]

To a solution of Succinic acid mono carboxy methyl ester (50 grams, 284.09 mmol) and Thionyl chloride (100 ml, 1.37 mol) was added DMF (0.5 ml), stirred at room temperature for 30 minutes and later heated to reflux for 16 hours. Excess Thionyl chloride was distilled under vacuum; Toluene (25 ml) added and distilled under vacuum to get the crude. The crude product was purified by high vacuum distillation to get pure Acid chloride (45 grams) as light yellow liquid.

### Example 51

### Synthesis of Dinitro Succinic Acid Glycolate

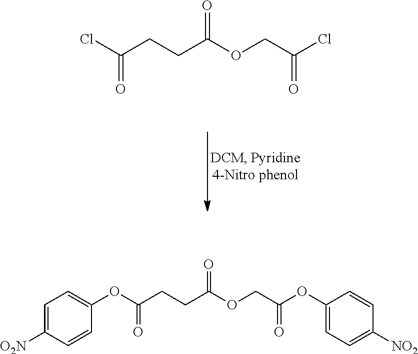 [SMILES: O=C(Cl)CCC(=O)OCC(=O)Cl ; O=C(CCC(=O)Oc1ccc([N+](=O)[O-])cc1)OCC(=O)Oc1ccc([N+](=O)[O-])cc1]

To a mixture of 4-Nitro phenol (53 grams, 380.99 mmol), Pyridine (43 ml, 531.65 mmol) in Dichloromethane (750 ml) at 0° C. was added Acid chloride (45 grams, 211.26 mmol) drop wise and later stirred at room temperature for 1 hour. The reaction mixture washed with water, 5% Sodium bicarbonate, 5% copper sulphate, water, dried over sodium sulphate, distilled off the solvent under reduced pressure and precipitated the crude compound by adding Diisopropyl ether, which was purified by recrystallising in a mixture of DMF:Methanol (2:6) to give pure Dinitro (50 grams) as white powder with a melting point of 84-86° C. The product was characterized by ^{I}H NMR (CDCl₃) δ 2.95 (m, 4H, CH₂X₂), 4.95 (s, 2H, CH₂), 7.30 (m, 4H, Ar), 8.30 (m, 4H, Ar).

### Example 52

### Synthesis of Succinic Acid Glycolate Diamine

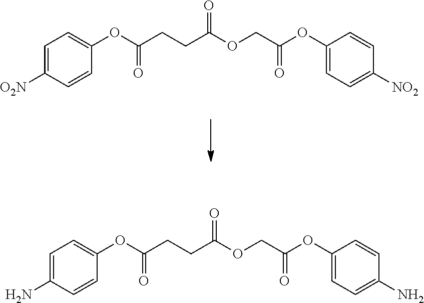 [SMILES: O=C(CCC(=O)Oc1ccc([N+](=O)[O-])cc1)OCC(=O)Oc1ccc([N+](=O)[O-])cc1 ; Nc1ccc(OC(=O)CCC(=O)OCC(=O)Oc2ccc(N)cc2)cc1]

Dinitro (30 grams, 71.77 mmol) was dissolved in DMF (150 ml) in a pressure vessel, 50% wet Palladium on carbon (5%, 6 grams) added and the mixture stirred under an atmosphere of hydrogen (5 Kg) for 1 hour. The catalyst was removed by filtration and precipitated by adding to cold water, dried and slurried in Ethyl acetate to get pure Diamine (17.5 grams) as off white powder with a melting point of 142-144° C. The product was characterized by ^{I}H NMR (DMSO-d₆) δ 2.80 (m, 4H, CH₂X₂), 4.90 (s, 2H, CH₂), 5.10 (bs, 2H, NH₂), 6.55 (m, 4H, Ar), 6.80 (m, 4H, Ar). Hydrolysis of 0.5 grams of diamine in a 50 ml buffer of pH 9 at 50° C. showed complete hydrolysis in 18 hours.

### Example 53

### Synthesis of Succinic Acid Glycolate Diisocyanate

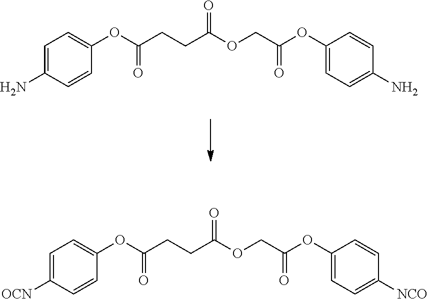

To a solution of Diamine (10 grams, 27.93 mmol) in dry 1,4-Dioxane (200 ml) under Nitrogen atmosphere at 0° C. was added a solution of Triphosgene (14 grams, 47.17 mmol) in 1,4-Dioxane (30 ml) in one lot and heated to 80° C. temperature for 2 hours. The condenser was then arranged for distillation and solvent removed by distillation at atmospheric pressure until the volume of the reaction mixture was reduced to approximately one third. Fresh dry Dioxane (50 ml) was added and distilled off the solvents under vacuum and this process of repeated distillation was continued for two more times by adding the same quantity of 1,4-Dioxane. The residue was dissolved in Toluene (50 ml), added charcoal (5 grams), filtered hot, precipitated the DiNCO by adding Hexane (300 ml), filtered, dried and packed in tight container to get 7 grams of pure Diisocyanate as a white powder with a melting point of 57-60° C. The product was characterized by ^{I}H NMR (CDCl₃) δ 2.90 (m, 4H, CH₂X₂), 4.80 (s, 2H, CH₂), 7.10 (m, 8H, Ar). The isocyanate was converted into a urethane with a melting point of 138-142° C. in order to determine rate of hydrolysis. Hydrolysis of 0.5 grams of diurethane in a 50 ml buffer of pH 9 at 50° C. showed complete hydrolysis in 20 hours.

### Example 54

### Synthesis of Polyurethane from Succinic Acid Glycolate Diisocyanate

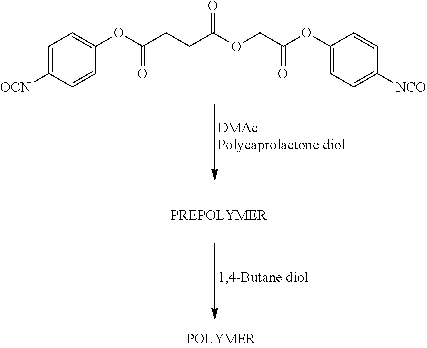

Into a clean flame dried 100 ml 3 neck round bottom flask maintained under nitrogen atmosphere in an oil bath maintained at 50° C. was added diisocyanate (5.1 grams) dissolved in DMAc (20 ml). Into this stirring solution was added polycaprolactone diol with an average molecular weight of 2000 (10 grams) and 3 drops of 10% stannous octoate solution. The solution was left for stirring at 60° C. for 4 hours. Into this solution of prepolymer was added 1,4-Butanediol (675 mg) dissolved in dimethylacetamide (10 ml). To this reaction mixture was added 3 drops of 10% stannous octoate solution. The reaction mixture was left for further stirring at 60° C. for 2 hour. The solution became very viscous. A small fraction of the reaction mixture was poured onto a petri dish and left overnight to generate film. A film of average strength was formed.

When ranges are used herein for physical properties, such as molecular weight, or chemical properties, such as chemical formulae, all combinations and subcombinations of ranges and specific embodiments therein are intended to be included.

Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. Absorbable isocyanates of Formula II and III*, 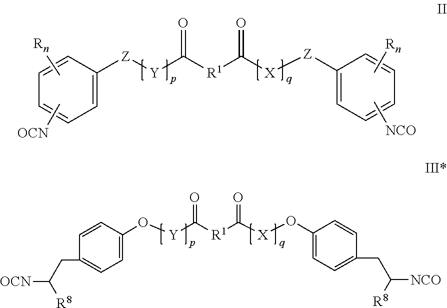
wherein:
each Y is independently:
—COCH₂O— (glycolic ester moiety);
—COCH(CH₃)O— (lactic ester moiety);
—COCH₂OCH₂CH₂O— (dioxanone ester moiety);
—COCH₂CH₂CH₂CH₂CH₂O— (caprolactone ester moiety);
—CO(CH₂)_{y}O— where y is one of the numbers 2, 3, 4, and 6-24 inclusive; or
—COCH₂O(CH₂CH₂O)ₘ— where m is integer between 2-24 inclusive; and,
each X is independently:
—OCH₂CO— (glycolic acid moiety);
—OCH(CH₃)CO— (lactic acid moiety);
—OCH₂CH₂OCH₂CO— (dioxanone moiety);
—OCH₂CH₂CH₂CH₂CH₂CO— (caprolactone moiety);
—O(CH₂)_{y}CO— or,
—O(CH₂CH₂O)ₘCH₂CO—;
R¹ is -(D)ᵣ-, wherein r is an integer between 1-24 inclusive, wherein if r is 1 or 2, D is —CH₂—, and if r is greater than 2, D is —CH₂— or —O—/—S— (oxygen or sulfur), wherein all individual carbons D are linked to one or two carbons, and all occurrences of —O—/—S— are linked to two carbons;
Z is —O—, —S— or —NH—;
p and q are independently integers between 0 and 6, and p+q is greater than or equal to 1;
each R is independently alkoxy, benzyloxy, aldehyde, halogen, carboxylic acid, NO₂ or NO₂—R⁷—, where R⁷ is lower alkyl;
R⁸ is H or —C(═O)OCH₃; and
n is 0 to 4.

2-3. (canceled)

4. An absorbable isocyanate according to claim 1, wherein the absorbable isocyanate is according to Formula II.

5. An absorbable isocyanate according to claim 4, wherein the absorbable isocyanate is compound 8-9 or 10, namely:

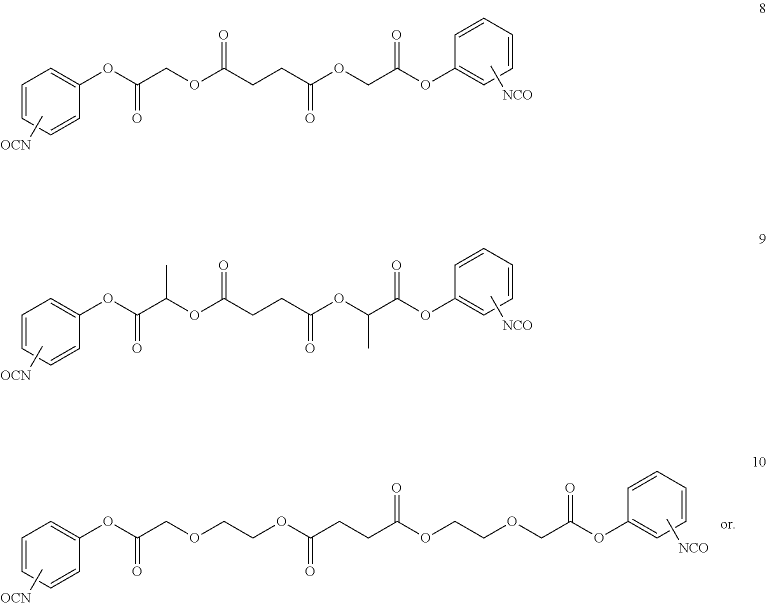

6. An absorbable isocyanate according to claim 1, wherein the absorbable isocyanate is according to Formula wherein R⁸ is H.

7. An absorbable isocyanate according to claim 6, wherein the absorbable isocyanate is compound 12-13 or 14, namely:

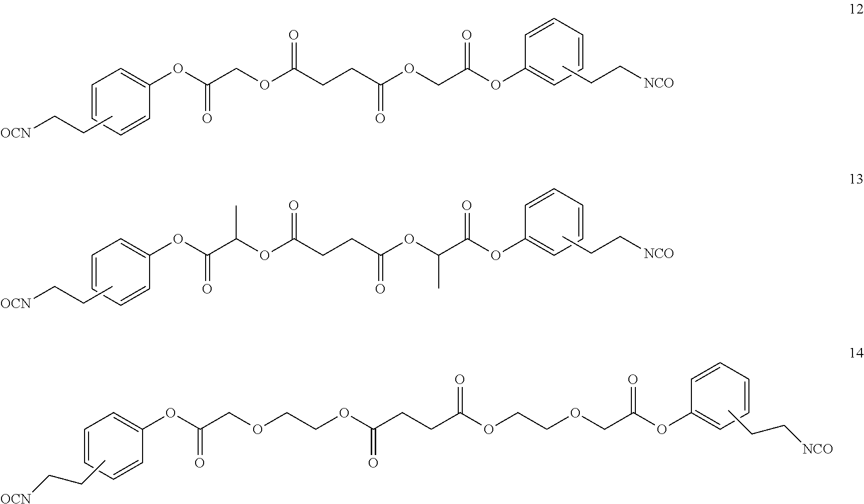

8. An absorbable isocyanate according to claim 1, wherein the absorbable isocyanate is according to Formula III*, wherein R⁸ is —C(═O)OCH₃.

9. An absorbable isocyanate according to claim 8, wherein the absorbable isocyanate is compound 15-16 or 17, namely:

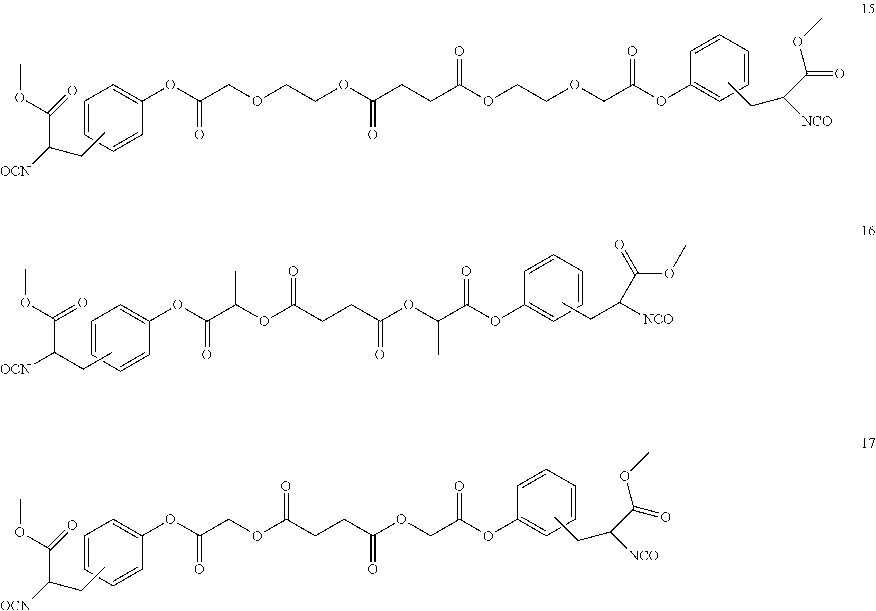

10-13. (canceled)

14. A surgical article or component thereof or polymeric carrier comprised of branched absorbable polyurethanes derived from isocyanates, chain extenders and polyols, wherein (A) and one or more of (B) to (C) applies:
(A) the isocyanates comprise a absorbable isocyanate of claim 1;
(B) the chain extenders comprise an absorbable chain extender of Formula V: 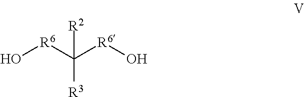
wherein:
R² and R³ are independently alkyl, —CO—(X)ₚ—O—R⁴ or —NH—(Y)_{q}—CO—R⁵, wherein alkyl is C6 to C20, or a biologically active substance, wherein in some embodiments no more than one of R² and R³ is alkyl;
R⁶ is -(D)_{r*}-, wherein r* is an integer between 1-24, wherein if r* is 1 or 2, D is —CH₂—, and if r* is greater than 2, D is —CH₂— or —O—/—S— (oxygen or sulfur), wherein all individual carbons D are linked to one or more carbons, and all occurrences of —O—/—S— are linked to two carbons;
R^{6′} is independently defined as is R⁶; and
R⁴ and R⁵ are each independently is alkyl, wherein alkyl is C1 to C20 (such as the alkyl moieties of stearic, oleic or linoleic acid), a biologically active substance, or a polyethylene glycol of molecular weight from 500 to 3000;
(C) the polyols comprise an absorbable polyol of Formula VI: 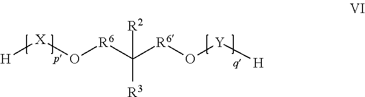
wherein p′ and q′ are independently 1-24.

15. A surgical article or component thereof according to claim 14, which is a tissue adhesive, stent, stent coating, wound film, covering or dressing, burn covering or dressing, surgical dressing, mesh, foam, highly porous foam, reticulated foam, drug device combination, medical device coating, tissue engineering scaffold, film, adhesion prevention barrier, implantable medical device, non-implantable medical device, controlled drug delivery device or system, or anastomosis ring.

16. A surgical article or component thereof according to claim 15, wherein a biologically active agent is physically embedded or dispersed into a polymer matrix of the comprised polymer.

17. A surgical article or component thereof according to claim 14, which is a suture, ligature, needle and suture combination, surgical clip, surgical staple, prosthesis, textile structure, coupling, tube, support, screw, pin

18. A surgical article or component thereof according to claim 14, which is a bone wax formulation or an adhesion prevention barrier.

19-21. (canceled)

22. A pharmaceutical composition comprising a polymeric carrier of claim 14 and a drug uniformly dispersed therein.

23. A pharmaceutical composition comprising a polymeric carrier of claim 19 and a drug uniformly dispersed therein.

24. An intermediate for forming an isocyanate of claim 1 or a polymerization monomer, which is an amine that is compound 1′-16′ or 17′.

25. A surgical article or component thereof or polymeric carrier, comprising a polymer formed by reacting a monomer of claim 24 with an isocyanate, carboxylic acid, activated carboxylic acid, or epoxide.

26. A surgical article or component thereof according to claim 25, which is a tissue adhesive, stent, stent coating, wound film, covering or dressing, burn covering or dressing, surgical dressing, mesh, foam, highly porous foam, reticulated foam, drug device combination, medical device coating, tissue engineering scaffold, film, adhesion prevention barrier, implantable medical device, non-implantable medical device, controlled drug delivery device or system, anastomosis ring, suture, ligature, needle and suture combination, surgical clip, surgical staple, prosthesis, textile structure, coupling, tube, support, screw, pin, bone wax formulation or an adhesion prevention barrier.
